(19) United States
(12) Patent Application Publication
(10) Pub. No.: US 2024/0368678 A1
(43) Pub. Date: Nov. 7, 2024

(54) METHODS AND COMPOSITIONS FOR SPATIAL ASSAY

(71) Applicant: 10x Genomics, Inc., Pleasanton, CA (US)

(72) Inventor: Octavian Marian BLOJU, Stockholm (SE)

(21) Appl. No.: 18/653,613

(22) Filed: May 2, 2024

Related U.S. Application Data
(60) Provisional application No. 63/463,863, filed on May 3, 2023.

(51) Int. Cl. C12Q 1/682; C12Q 1/6876

(52) U.S. Cl. CPC C12Q 1/682; C12Q 1/6876; C12Q 2600/166

(57) ABSTRACT

The present disclosure relates in some aspects to methods, compositions, and kits for profiling biological targets in a sample. In some embodiments, a biological sample is analyzed by capturing nucleic acids of the biological sample using oligonucleotides immobilized on a substrate configured to align with the biological sample, hybridizing probes to the nucleic acids or to complements thereof, and detecting the probes or products or complexes thereof at locations on the substrate, thereby detecting the nucleic acids at the corresponding locations in the biological sample.

### CROSS-REFERENCE TO RELATED APPLICATION

This application claims priority to U.S. Provisional Application No. 63/463,863, filed on May 3, 2023, entitled “METHODS AND COMPOSITIONS FOR SPATIAL ASSAY”, which is herein incorporated by reference in its entirety for all purposes.

### FIELD

The present disclosure relates in some aspects to methods, compositions, and systems for analysis of spatial information of analytes in biological samples.

### BACKGROUND

Profiling biological targets in a sample, such as genomic, transcriptomic, or proteomic profiling of cells, are essential for many purposes, such as understanding the molecular basis of cell identity and developing treatment for diseases. Microscopy imaging, which can resolve multiple analytes in a sample, provides valuable information such as analyte abundance and spatial information of analytes in situ. However, existing in situ hybridization and imaging-based approaches may suffer from low efficiency, but the potential value of such in-tissue analysis could be enormous. Therefore, there is a need for new and improved methods for analyzing analytes and their relative spatial locations in a biological sample.

### SUMMARY

In some aspects, the present application provides new and improved methods, compositions, and kits for profiling analytes in a sample using a substrate comprising capture agents as well as one or more stabilization agents and/or one or more interspersing agents.

In some aspects, the provided methods comprise capturing target nucleic acids using immobilized oligonucleotide molecules (e.g., capture agents, and stabilization agents and/or interspersing agents) on a substrate that is aligned with the biological sample, generating a product or complex of each captured target nucleic acid or a complement thereof on the substrate, and detecting optical signals associated with the products or complexes on the substrate. For example, the products can be generated using rolling circle amplification (RCA) and the rolling circle amplification products (RCPs) are detected on a substrate rather than in a biological sample, allowing for more efficient reactions and washes and reducing background signal such as autofluorescence. In some aspects, data obtained from analysis of the RCPs on the substrate is superimposed with data obtained from imaging the biological sample to correlate the locations of the detected RCPs with locations of target nucleic acids in the biological sample as well as other data, e.g., tissue morphology or other features of the sample.

In some embodiments, provided herein is a method for analyzing a biological sample, comprising: a) contacting the biological sample comprising cells with a substrate, wherein: the biological sample comprises a plurality of target nucleic acids, the substrate comprises a plurality of capture agents and a plurality of stabilization agents, and each capture agent is a nucleic acid comprising a capture domain capable of capturing a target nucleic acid of the plurality of target nucleic acids; b) providing conditions to allow the capture agents to capture the target nucleic acids; c) contacting the substrate with a probe or probe set that binds to the captured target nucleic acid or a complement thereof; d) generating an amplification product associated with the probe or probe set, wherein the amplification product comprises a plurality of stabilizing sequences complementary to a sequence of each of the plurality of stabilization agents; and e) detecting a signal associated with the amplification product on the substrate, wherein the location of the signal on the substrate corresponds to the location of the target nucleic acid in the biological sample.

In some embodiments, provided herein is a method for analyzing a biological sample, comprising: a) contacting the biological sample with a substrate, wherein: the biological sample comprises a plurality of target nucleic acids, the substrate comprises a plurality of capture agents and a plurality of interspersing agents immobilized at interspersed locations on the substrate, and each capture agent comprises a capture domain capable of capturing a target nucleic acid of the plurality of target nucleic acids; b) providing conditions to allow the capture agents to capture the target nucleic acids, under which conditions the interspersing agents do not capture the target nucleic acids; c) contacting the substrate with a probe or probe set that binds to the captured target nucleic acid or a complement thereof; and d) detecting a signal associated with the probe or probe set or a complex or product thereof on the substrate, wherein the location of the signal on the substrate corresponds to the location of the target nucleic acid in the biological sample.

In some embodiments, provided herein is a method for analyzing a biological sample, comprising: a) contacting the biological sample comprising cells with a substrate, wherein: the biological sample comprises a plurality of target nucleic acids, the substrate comprises a plurality of capture agents and a plurality of stabilization agents, and each capture agent is a nucleic acid comprising a capture domain capable of capturing a ligated probe generated from ligation of oligonucleotide probes hybridized to a target nucleic acid of the plurality of target nucleic acids; b) providing conditions to allow the capture agents to capture the ligated probes; c) contacting the substrate with a probe or probe set that binds to the captured ligated probe or a complement thereof; d) generating an amplification product associated with the probe or probe set, wherein the amplification product comprises a plurality of stabilizing sequences complementary to a sequence of each of the plurality of stabilization agents; and e) detecting a signal associated with the amplification product on the substrate, wherein the location of the signal on the substrate corresponds to the location of the target nucleic acid in the biological sample.

In some embodiments, provided herein is a method for analyzing a biological sample, comprising: a) contacting the biological sample comprising cells with a substrate, wherein: the biological sample comprises a plurality of target nucleic acids, the substrate comprises a plurality of capture agents and a plurality of stabilization agents, and each capture agent is a nucleic acid comprising a capture domain capable of capturing a ligated probe generated from ligation of oligonucleotide probes hybridized to a target nucleic acid of the plurality of target nucleic acids; b) providing conditions to allow the capture agents to capture the ligated probes; c) extending the capture agent by a polymerase using the captured ligated probe as a template, thereby generating an extended capture agent comprising a complement of the captured ligated probe; d) contacting the substrate with a probe or probe set that binds to the complement of the captured ligated probe; e) generating an amplification product associated with the probe or probe set, wherein the amplification product comprises a plurality of stabilizing sequences complementary to a sequence of each of the plurality of stabilization agents; and f) detecting a signal associated with the amplification product on the substrate, wherein the location of the signal on the substrate corresponds to the location of the target nucleic acid in the biological sample.

In some embodiments, provided herein is a method for analyzing a biological sample, comprising: a) contacting the biological sample with a substrate, wherein: the biological sample comprises a plurality of target nucleic acids, the substrate comprises a plurality of capture agents and a plurality of interspersing agents immobilized at interspersed locations on the substrate, and each capture agent comprises a capture domain capable of capturing a ligated probe generated from ligation of oligonucleotide probes hybridized to a target nucleic acid of the plurality of target nucleic acids; b) providing conditions to allow the capture agents to capture the ligated probes, under which conditions the interspersing agents do not capture the ligated probes; c) contacting the substrate with a probe or probe set that binds to the captured ligated probe or a complement thereof; and d) detecting a signal associated with the probe or probe set or a complex or product thereof on the substrate, wherein the location of the signal on the substrate corresponds to the location of the target nucleic acid in the biological sample.

In some embodiments, provided herein is a method for analyzing a biological sample, comprising: a) contacting the biological sample with a substrate, wherein: the biological sample comprises a plurality of target nucleic acids, the substrate comprises a plurality of capture agents and a plurality of interspersing agents immobilized at interspersed locations on the substrate, and each capture agent comprises a capture domain capable of capturing a ligated probe generated from ligation of oligonucleotide probes hybridized to a target nucleic acid of the plurality of target nucleic acids; b) providing conditions to allow the capture agents to capture the ligated probes, under which conditions the interspersing agents do not capture the ligated probes; c) extending the capture agent by a polymerase using the captured ligated probe as a template, thereby generating an extended capture agent comprising a complement of the captured ligated probe; d) contacting the substrate with a probe or probe set that binds to the complement of the captured ligated probe; and e) detecting a signal associated with the probe or probe set or a complex or product thereof on the substrate, wherein the location of the signal on the substrate corresponds to the location of the target nucleic acid in the biological sample.

In any one or more of the embodiments herein, the biological sample is immobilized on the substrate. In any one or more of the embodiments herein, the substrate is a first substrate and the biological sample is immobilized on a second substrate and the first and second substrates are aligned with each other during the contacting of the biological sample with the substrate, e.g., in a). In any one or more of the embodiments herein, the method comprises applying one or more spacers to the first substrate and/or the second substrate to maintain a minimum spacing between the first substrate after or before aligning the first and second substrates. In any one or more of the embodiments herein, the minimum space is less than 50 microns, less than 25 microns, or less than 20 microns. In any one or more of the embodiments herein, the method comprises applying a permeabilization reagent to the first substrate and/or the second substrate.

In any one or more of the embodiments herein, the method or kit does not need to but may comprise a step or a reagent for a proximity ligation assay. In any one or more of the embodiments herein, the method or kit does not need to but may comprise a step or a reagent for detecting a target protein using one or more binders that bind to the protein. In any one or more of the embodiments herein, the method or kit may comprise a step or a device (e.g., a device as shown in FIG. 4, FIGS. 5A-5B, or FIGS. 6A-6C) to allow the capture agents to capture the target nucleic acids and/or the ligated probes.

In some aspects, provided herein is a method for analyzing a biological sample, comprising: a) contacting the biological sample with a substrate, wherein: the biological sample comprises a plurality of target nucleic acids, the substrate comprises a plurality of capture agents and a plurality of stabilization agents, and each capture agent comprises a capture domain capable of capturing a target nucleic acid of the plurality of target nucleic acids; b) providing conditions to allow the capture agents to capture the target nucleic acids; c) contacting the substrate with a probe or probe set that binds to the captured target nucleic acid or a complement thereof; d) generating an amplification product associated with the probe or probe set, wherein the amplification product comprises a plurality of stabilizing sequences complementary to a sequence of each of the plurality of stabilization agents; and e) detecting a signal associated with the amplification product, thereby detecting the target nucleic acid in the biological sample.

In any one or more of the embodiments disclosed herein, the biological sample is immobilized on the substrate. In any one or more of the embodiments disclosed herein, the substrate is a first substrate and the biological sample is immobilized on a second substrate. In any one or more of the embodiments disclosed herein, the first and second substrates are configured to be aligned with each other. In any one or more of the embodiments disclosed herein, the plurality of capture agents are in a capture area of the first substrate, the plurality of target nucleic acids are in a sample area of the biological sample, and the capture area and the sample area is configured to be aligned with each other. In any one or more of the embodiments disclosed herein, the method comprises aligning the first and second substrates and migrating the plurality of target nucleic acids from the biological sample to the substrate and the plurality of capture agents thereon.

In any one or more of the embodiments disclosed herein, the plurality of capture agents and/or the plurality of stabilization agents are directly or indirectly immobilized on the substrate. In any one or more of the embodiments disclosed herein, the plurality of capture agents and/or the plurality of stabilization agents are covalently or non-covalently immobilized on the substrate. In any one or more of the embodiments disclosed herein, the plurality of capture agents and/or the plurality of stabilization agents are randomly distributed on the substrate.

In any one or more of the embodiments disclosed herein, the plurality of capture agents and/or the plurality of stabilization agents form an oligonucleotide lawn on the substrate. In some embodiments, the plurality of capture agents and the plurality of stabilization agents are not distributed in a pattern of discrete features on the substrate. In any one or more of the embodiments disclosed herein, the plurality of stabilization agents are interspersed among the plurality of capture agents which form a lawn of oligonucleotides uniformly distributed on the substrate. In any one or more of the embodiments disclosed herein, the plurality of capture agents are interspersed among the plurality of stabilization agents which form a lawn of oligonucleotides uniformly distributed on the substrate.

In any one or more of the embodiments disclosed herein, the ratio between molecules of the plurality of capture agents and molecules of the plurality of stabilization agents on the substrate or in a capture area thereof is greater than 100:1, about 100:1, about 50:1, about 20:1, about 10:1, about 5:1, about 2:1, about 1:1, about 1:2, about 1:5, about 1:10, about 1:20, about 1:50, about 1:100, or less than 1:100. In any one or more of the embodiments disclosed herein, prior to immobilization of the plurality of capture agents and the plurality of stabilization agents on the substrate, the method comprises mixing molecules of the plurality of capture agents and molecules of the plurality of stabilization agents at a ratio of greater than 100:1, about 100:1, about 50:1, about 20:1, about 10:1, about 5:1, about 2:1, about 1:1, about 1:2, about 1:5, about 1:10, about 1:20, about 1:50, about 1:100, or smaller than 1:100. In any one or more of the embodiments disclosed herein, the density of molecules of the plurality of capture agents and molecules of the plurality of stabilization agents on the substrate or in a capture area on the substrate is between about 1 and about 10 picomoles per 10 μm² area.

In some embodiments, the method does not comprise arranging molecules of the plurality of capture agents and/or molecules of the plurality of stabilization agents in distinctive features on the substrate. In some embodiments, the method does not comprise printing molecules of the plurality of capture agents and/or molecules of the plurality of stabilization agents in array spots on the substrate.

In any one or more of the embodiments disclosed herein, molecules of the plurality of capture agents and/or molecules of the plurality of stabilization agents are uniformly distributed on the substrate or in a capture area on the substrate. In any one or more of the embodiments disclosed herein, one or more molecules of the plurality of capture agents and/or the plurality of stabilization agents each comprises a spatial barcode corresponding to the location of the capture agent or stabilization agent on the substrate. In some embodiments, none of the plurality of capture agents and the plurality of stabilization agents comprises a spatial barcode corresponding to the location of the capture agent or stabilization agent on the substrate.

In any one or more of the embodiments disclosed herein, the capture domains of two or more of the plurality of capture agents comprise different nucleic acid sequences. In any one or more of the embodiments disclosed herein, the capture domain of each of the plurality of capture agents is complementary to a different target nucleic acid sequence. In any one or more of the embodiments disclosed herein, the capture domains of two or more of the plurality of capture agents comprise a common nucleic acid sequence. In any one or more of the embodiments disclosed herein, the capture domain of each of the plurality of capture agents can comprise the common nucleic acid sequence. In any one or more of the embodiments disclosed herein, the capture domain of each of the plurality of capture agents can comprise a poly(dT) sequence. In any one or more of the embodiments disclosed herein, the poly(dT) sequence is about 10, about 15, about 20, about 25, about 30, or more than 30 nucleotides in length. In any one or more of the embodiments disclosed herein, each of the plurality of target nucleic acids comprise a poly(A) sequence. In any one or more of the embodiments disclosed herein, the plurality of target nucleic acids comprise mRNA of different genes.

In any one or more of the embodiments disclosed herein, two or more of the plurality of stabilization agents comprise different nucleic acid sequences. In any one or more of the embodiments disclosed herein, two or more of the plurality of stabilization agents comprise a common nucleic acid sequence. In any one or more of the embodiments disclosed herein, each of the plurality of stabilization agents comprise the common nucleic acid sequence. In any one or more of the embodiments disclosed herein, the common nucleic acid sequence in the stabilization agents is complementary to the plurality of stabilizing sequences in the amplification product.

In some embodiments, the plurality of stabilization agents do not comprise a capture domain capable of capturing a target nucleic acid of the plurality of target nucleic acids. In some embodiments, the plurality of stabilization agents do not comprise a sequence complementary to a target nucleic acid of the plurality of target nucleic acids. In some embodiments, the plurality of stabilization agents do not comprise a poly(dT) sequence of about 10, about 15, about 20, about 25, about 30, or more than 30 nucleotides in length. In some embodiments, the plurality of stabilization agents do not comprise a poly(dT) sequence. In some embodiments, the plurality of stabilization agents do not comprise a sequence of more than 10, 20, or 30 consecutive nucleotides complementary to a target nucleic acid of the plurality of target nucleic acids.

In any one or more of the embodiments disclosed herein, the conditions provided to allow the capture agents to capture the target nucleic acids comprise releasing the target nucleic acids from the biological sample. In any one or more of the embodiments disclosed herein, the conditions provided to allow the capture agents to capture the target nucleic acids comprise migrating the target nucleic acids towards the substrate. In any one or more of the embodiments disclosed herein, the conditions provided to allow the capture agents to capture the target nucleic acids comprise contacting the target nucleic acids with the capture agents and the stabilization agents, such that target nucleic acids captured at adjacent locations are spaced from one another by one or more stabilization agents.

In any one or more of the embodiments disclosed herein, the method comprises generating the complement of the captured target nucleic acid at the location of the captured target nucleic acid on the substrate. In any one or more of the embodiments disclosed herein, the method comprises extending the capture domain or a portion thereof by a polymerase using the captured target nucleic acid as a template. In any one or more of the embodiments disclosed herein, the method comprises removing the captured target nucleic acid or a portion thereof from the substrate, e.g., after generating the complement of the captured target nucleic acid. In any one or more of the embodiments disclosed herein, the removing comprises digesting the captured target nucleic acid using an enzyme. In any one or more of the embodiments disclosed herein, the captured target nucleic acid is RNA, the complement is cDNA, and the enzyme is an RNase H. In any one or more of the embodiments disclosed herein, the removing comprises denaturing a duplex formed by the captured target nucleic acid and the complement, and the complement can remain immobilized on the substrate via the capture agent after the denaturation. In any one or more of the embodiments disclosed herein, the method comprises generating a complement of the complement of the captured target nucleic acid. In any one or more of the embodiments disclosed herein, the method comprises removing the complement of the complement from the substrate for sequence analysis.

In any one or more of the embodiments disclosed herein, the probe or probe set comprises a 3′ overhang and/or a 5′ overhang upon hybridization to the captured target nucleic acid or complement thereof. In any one or more of the embodiments disclosed herein, the probe or probe set is a circular probe or circularizable probe or probe set. In any one or more of the embodiments disclosed herein, the circularizable probe or probe set is ligated using the captured target nucleic acid or complement thereof as a template, with or without gap filling prior to the ligation. In any one or more of the embodiments disclosed herein, the circularizable probe or probe set is ligated using a splint other than the captured target nucleic acid or complement thereof as a template, with or without gap filling prior to the ligation. In any one or more of the embodiments disclosed herein, the splint hybridizes to the captured target nucleic acid or complement thereof.

In any one or more of the embodiments disclosed herein, the probe or probe set comprises one or more barcode regions. In any one or more of the embodiments disclosed herein, the amplification product is a rolling circle amplification product (RCP), e.g., an RCP comprising multiple copies of the complement of a barcode region in the probe or probe set. In any one or more of the embodiments disclosed herein, the 3′ end of the complement of the captured target nucleic acid can be blocked from exonuclease digestion and primer extension, and the RCP can be generated using a primer distinct from the complement of the captured target nucleic acid. In any one or more of the embodiments disclosed herein, the 3′ end of the complement of the captured target nucleic acid can be unblocked from exonuclease digestion and primer extension, and the RCP can be generated using the complement or a portion thereof as a primer.

In any one or more of the embodiments disclosed herein, the RCP is hybridized to one or more of the plurality of stabilization agents via one or more of the stabilizing sequences in the RCP. In any one or more of the embodiments disclosed herein, the method comprises crosslinking the RCP to the stabilization agents and/or to the capture agents after the RCP hybridizing to the one or more stabilization agents. In some embodiments, the method does not comprise crosslinking the RCP to the stabilization agents and/or to the capture agents.

In any one or more of the embodiments disclosed herein, the target nucleic acid can be a first target nucleic acid and the probe or probe set can be a first probe or probe set, and the method comprises: f) contacting the substrate with a second probe or probe set that binds to a second captured target nucleic acid or a complement thereof, wherein the first and second target nucleic acids are different; g) generating a second amplification product associated with the second probe or probe set, wherein the second amplification product comprises a plurality of stabilizing sequences complementary to a sequence of each of the plurality of stabilization agents; and h) detecting a signal associated with the second amplification product, thereby detecting the second target nucleic acid in the biological sample.

In any one or more of the embodiments disclosed herein, the method comprises removing the first probe or probe set, the first amplification product, and/or one or more nucleic acid probes that bind to the first amplification product for detecting the signal associated therewith, prior to contacting the substrate with the second probe or probe set. In any one or more of the embodiments disclosed herein, the removing comprises denaturation using heat denaturation, a denaturing agent, enzymatic cleavage, and/or chemical cleavage. In any one or more of the embodiments disclosed herein, the nucleic acid probes hybridizes to barcode sequences in the RCP.

In some aspects, provided herein is a method for analyzing a biological sample, comprising: a) contacting the biological sample with a substrate, wherein: the biological sample comprises a plurality of target nucleic acids, the substrate comprises a plurality of capture agents and a plurality of interspersing agents immobilized at interspersed locations on the substrate, and each capture agent comprises a capture domain capable of capturing a target nucleic acid of the plurality of target nucleic acids; b) providing conditions to allow the capture agents to capture the target nucleic acids, under which conditions the interspersing agents do not capture the target nucleic acids; c) contacting the substrate with a probe or probe set that binds to the captured target nucleic acid or a complement thereof; and d) detecting a signal associated with the probe or probe set or a complex or product thereof, thereby detecting the target nucleic acid in the biological sample.

In any one or more of the embodiments disclosed herein, the biological sample is immobilized on the substrate. In any one or more of the embodiments disclosed herein, the substrate can be a first substrate and the biological sample can be immobilized on a second substrate. In any one or more of the embodiments disclosed herein, the first and second substrates are configured to be aligned with each other. In any one or more of the embodiments disclosed herein, the plurality of capture agents can be in a capture area of the first substrate, the plurality of target nucleic acids can be in a sample area of the biological sample, and the capture area and the sample area can be configured to be aligned with each other. In any one or more of the embodiments disclosed herein, the method comprises aligning the first and second substrates and migrating the plurality of target nucleic acids from the biological sample to the plurality of capture agents.

In any one or more of the embodiments disclosed herein, the plurality of capture agents and/or the plurality of interspersing agents are directly or indirectly immobilized on the substrate. In any one or more of the embodiments disclosed herein, the plurality of capture agents and/or the plurality of interspersing agents are covalently or non-covalently immobilized on the substrate. In any one or more of the embodiments disclosed herein, the plurality of capture agents and/or the plurality of interspersing agents are randomly distributed on the substrate.

In any one or more of the embodiments disclosed herein, the plurality of capture agents and the plurality of interspersing agents form an oligonucleotide lawn on the substrate. In some embodiments, the plurality of capture agents and/or the plurality of interspersing agents are not distributed in a pattern of discrete features on the substrate. In any one or more of the embodiments disclosed herein, the plurality of interspersing agents are interspersed among the plurality of capture agents which form a lawn of oligonucleotides uniformly distributed on the substrate. In any one or more of the embodiments disclosed herein, the plurality of capture agents are interspersed among the plurality of interspersing agents which form a lawn of oligonucleotides uniformly distributed on the substrate.

In any one or more of the embodiments disclosed herein, the ratio between molecules of the plurality of capture agents and molecules of the plurality of interspersing agents on the substrate is greater than 100:1, about 100:1, about 50:1, about 20:1, about 10:1, about 5:1, about 2:1, about 1:1, about 1:2, about 1:5, about 1:10, about 1:20, about 1:50, about 1:100, or less than 1:100. In any one or more of the embodiments disclosed herein, prior to immobilization of the plurality of capture agents and the plurality of interspersing agents on the substrate, the method comprises mixing molecules of the plurality of capture agents and molecules of the plurality of interspersing agents at a ratio of greater than 100:1, about 100:1, about 50:1, about 20:1, about 10:1, about 5:1, about 2:1, about 1:1, about 1:2, about 1:5, about 1:10, about 1:20, about 1:50, about 1:100, or smaller than 1:100. In any one or more of the embodiments disclosed herein, the density of molecules of the plurality of capture agents and molecules of the plurality of interspersing agents on the substrate or in a capture area on the substrate is between about 1 and about 10 picomoles per 10 μm² area.

In some embodiments, the method does not comprise placing molecules of the plurality of capture agents and/or molecules of the plurality of interspersing agents in distinctive features on the substrate. In some embodiments, the method does not comprise printing molecules of the plurality of capture agents and/or molecules of the plurality of interspersing agents in array spots on the substrate.

In any one or more of the embodiments disclosed herein, molecules of the plurality of capture agents and/or molecules of the plurality of interspersing agents are uniformly distributed on the substrate or in a capture area on the substrate. In any one or more of the embodiments disclosed herein, one or more molecules of the plurality of capture agents and/or the plurality of interspersing agents each comprise a spatial barcode corresponding to the location of the capture agent or interspersing agent on the substrate. In some embodiments, none of the plurality of capture agents and the plurality of interspersing agents comprises a spatial barcode corresponding to the location of the capture agent or interspersing agent on the substrate.

In any one or more of the embodiments disclosed herein, the capture domains of two or more of the plurality of capture agents comprise different nucleic acid sequences. In any one or more of the embodiments disclosed herein, the capture domain of each of the plurality of capture agents can be complementary to a different target nucleic acid sequence. In any one or more of the embodiments disclosed herein, the capture domains of two or more of the plurality of capture agents can comprise a common nucleic acid sequence. In any one or more of the embodiments disclosed herein, the capture domain of each of the plurality of capture agents comprises the common nucleic acid sequence. In any one or more of the embodiments disclosed herein, the capture domain of each of the plurality of capture agents can comprise a poly(dT) sequence. In any one or more of the embodiments disclosed herein, the poly(dT) sequence can be about 10, about 15, about 20, about 25, about 30, or more than 30 nucleotides in length. In any one or more of the embodiments disclosed herein, each of the plurality of target nucleic acids can comprise a poly(A) sequence. In any one or more of the embodiments disclosed herein, the plurality of target nucleic acids comprise mRNA of different genes.

In any one or more of the embodiments disclosed herein, two or more of the plurality of interspersing agents can comprise different nucleic acid sequences. In any one or more of the embodiments disclosed herein, two or more of the plurality of interspersing agents comprise a common nucleic acid sequence. In any one or more of the embodiments disclosed herein, each of the plurality of interspersing agents comprise the common nucleic acid sequence. In any one or more of the embodiments disclosed herein, the common nucleic acid sequence in the interspersing agents are complementary to a plurality of stabilizing sequences in the complex or product (e.g., RCP) of the probe or probe set.

In some embodiments, the plurality of interspersing agents do not comprise a capture domain capable of capturing a target nucleic acid of the plurality of target nucleic acids. In some embodiments, the plurality of interspersing agents do not comprise a sequence complementary to a target nucleic acid of the plurality of target nucleic acids. In some embodiments, the plurality of interspersing agents do not comprise a poly(dT) sequence of about 10, about 15, about 20, about 25, about 30, or more than 30 nucleotides in length. In some embodiments, the plurality of interspersing agents do not comprise a poly(dT) sequence. In some embodiments, the plurality of interspersing agents do not comprise a sequence of more than 10, 20, or 30 consecutive nucleotides complementary to a target nucleic acid of the plurality of target nucleic acids.

In any one or more of the embodiments disclosed herein, the conditions provided to allow the capture agents to capture the target nucleic acids comprise releasing the target nucleic acids from the biological sample. In any one or more of the embodiments disclosed herein, the conditions provided to allow the capture agents to capture the target nucleic acids comprise migrating the target nucleic acids towards the substrate. In any one or more of the embodiments disclosed herein, the conditions provided to allow the capture agents to capture the target nucleic acids comprise contacting the target nucleic acids with the capture agents and the interspersing agents, such that target nucleic acids captured at adjacent locations are spaced from one another by one or more interspersing agents.

In any one or more of the embodiments disclosed herein, the method comprises generating the complement of the captured target nucleic acid at the location of the captured target nucleic acid on the substrate. In any one or more of the embodiments disclosed herein, the method comprises extending the capture domain or a portion thereof by a polymerase using the captured target nucleic acid as a template. In any one or more of the embodiments disclosed herein, the method comprises removing the captured target nucleic acid or a portion thereof from the substrate, e.g., after generating the complement of the captured target nucleic acid. In any one or more of the embodiments disclosed herein, the removing comprises digesting the captured target nucleic acid using an enzyme. In any one or more of the embodiments disclosed herein, the captured target nucleic acid is RNA, the complement is cDNA, and the enzyme is an RNase H. In any one or more of the embodiments disclosed herein, the removing comprises denaturing a duplex formed by the captured target nucleic acid and the complement, and the complement can remain immobilized on the substrate via the capture agent after the denaturation. In any one or more of the embodiments disclosed herein, the method comprises generating a complement of the complement of the captured target nucleic acid. In any one or more of the embodiments disclosed herein, the method comprises removing the complement of the complement from the substrate for sequence analysis.

In any one or more of the embodiments disclosed herein, the probe or probe set comprises a 3′ overhang and/or a 5′ overhang upon hybridization to the captured target nucleic acid or complement thereof. In any one or more of the embodiments disclosed herein, the probe or probe set can be a circular probe or circularizable probe or probe set. In any one or more of the embodiments disclosed herein, the circularizable probe or probe set is ligated using the captured target nucleic acid or complement thereof as a template, with or without gap filling prior to the ligation. In any one or more of the embodiments disclosed herein, the circularizable probe or probe set is ligated using a splint other than the captured target nucleic acid or complement thereof as a template, with or without gap filling prior to the ligation. In any one or more of the embodiments disclosed herein, the splint hybridizes to the captured target nucleic acid or complement thereof.

In any one or more of the embodiments disclosed herein, the probe or probe set comprises one or more barcode regions. In any one or more of the embodiments disclosed herein, the complex or product of the probe or probe set can comprise: a rolling circle amplification product (RCP); a complex comprising an initiator and an amplifier for hybridization chain reaction (HCR); a complex comprising an initiator and an amplifier for linear oligonucleotide hybridization chain reaction (LO-HCR); a primer exchange reaction (PER) product; and/or a complex comprising a pre-amplifier and an amplifier for branched DNA (bDNA). In any one or more of the embodiments disclosed herein, the 3′ end of the complement of the captured target nucleic acid can be blocked from exonuclease digestion and primer extension, and the RCP can be generated using a primer distinct from the complement of the captured target nucleic acid. In any one or more of the embodiments disclosed herein, the 3′ end of the complement of the captured target nucleic acid can be unblocked from exonuclease digestion and primer extension, and the RCP can be generated using the complement or a portion thereof as a primer.

In some embodiments, the RCP does not hybridize to one or more of the plurality of interspersing agents. In some embodiments, the RCP hybridizes to one or more of the plurality of interspersing agents. In some embodiments, the RCP comprises a plurality of sequences each complementary to one of the plurality of interspersing agents. In some embodiments, the method comprises crosslinking the RCP to the interspersing agents and/or to the capture agents after the RCP hybridizing to the one or more interspersing agents.

In any one or more of the embodiments disclosed herein, the RCP is detected using nucleic acid probes that hybridize to barcode sequences in the RCP.

In any one or more of the embodiments disclosed herein, the target nucleic acid can be a first target nucleic acid and the probe or probe set can be a first probe or probe set, and the method comprises: e) contacting the substrate with a second probe or probe set that binds to a second captured target nucleic acid or a complement thereof, wherein the first and second target nucleic acids are different; and f) detecting a signal associated with the second probe or probe set or a complex or product thereof, thereby detecting the second target nucleic acid in the biological sample.

In any one or more of the embodiments disclosed herein, the method comprises removing the first probe or probe set, the complex or product of the first probe or probe set, and/or one or more nucleic acid probes that bind to the first probe or probe set or the complex or product thereof for detecting the signal associated therewith, prior to contacting the substrate with the second probe or probe set. In any one or more of the embodiments disclosed herein, the removing comprises denaturation using heat denaturation, a denaturing agent, enzymatic cleavage, and/or chemical cleavage. In any one or more of the embodiments disclosed herein, the nucleic acid probes hybridizes to barcode sequences in the complex or product (e.g., RCP).

In any one or more of the embodiments disclosed herein, the probe or probe set is a circular or circularizable probe or probe set and the product can be a rolling circle amplification product (RCP). In any one or more of the embodiments disclosed herein, the RCP is detected using a detectably labeled probe that hybridizes to the RCP. In any one or more of the embodiments disclosed herein, the RCP is detected using an intermediate probe that hybridizes to the RCP, and a detectably labeled probe that hybridizes to the intermediate probe. In any one or more of the embodiments disclosed herein, the RCP is detected using sequential hybridization of multiple probe set, each probe set comprising an intermediate probe that hybridizes to the RCP and a detectably labeled probe that hybridizes to the intermediate probe.

In any one or more of the embodiments disclosed herein, the detecting comprises imaging the substrate using fluorescent microscopy. In any one or more of the embodiments disclosed herein, signals are detected at multiple locations on the substrate, wherein each signal is associated with a particular probe or probe set targeting a particular captured target nucleic acid, thereby detecting the plurality of target nucleic acids at locations in the biological sample. In any one or more of the embodiments disclosed herein, the signals detected at the multiple locations on the substrate are optically resolvable from one another and successfully decoded. In any one or more of the embodiments disclosed herein, the multiple locations on the substrate are spaced out from one another by one or more stabilization agents and/or interspersing agents on the substrate.

In any one or more of the embodiments disclosed herein, the plurality of target nucleic acids comprise a cellular RNA, a cDNA, a genomic DNA, and/or a reporter oligonucleotide. In any one or more of the embodiments disclosed herein, the reporter oligonucleotide is conjugated to a binding moiety that binds to a non-nucleic acid analyte. In any one or more of the embodiments disclosed herein, the binding moiety comprises an antibody or epitope binding fragment thereof.

In any one or more of the embodiments disclosed herein, the biological sample comprises a cell or tissue sample. In any one or more of the embodiments disclosed herein, the biological sample is a tissue section. In any one or more of the embodiments disclosed herein, the biological sample is a fresh frozen tissue section. In any one or more of the embodiments disclosed herein, the biological sample is a paraffin embedded formalin fixed (FFPE) tissue section.

In any one or more of the embodiments disclosed herein, the biological sample is permeabilized to allow the capture agents to capture the target nucleic acids. In any one or more of the embodiments disclosed herein, the method comprises staining and imaging the biological sample. In any one or more of the embodiments disclosed herein, the staining and imaging is performed prior to providing conditions to allow the capture agents to capture the target nucleic acids. In any one or more of the embodiments disclosed herein, the biological sample is stained with a nuclear stain, a histological stain, and/or an immunologic stain.

In some aspects, provided herein is a kit for analyzing a biological sample, comprising: a substrate comprising a plurality of capture agents and a plurality of stabilization agents immobilized thereon, wherein each capture agent comprises a capture domain capable of capturing a target nucleic acid of a plurality of target nucleic acids; and a plurality of probes or probe sets, wherein each probe or probe set binds to a target nucleic acid of the plurality of target nucleic acids or a complement of the target nucleic acid, and the probes or probe sets for different target nucleic acids share a common sequence, wherein the plurality of stabilization agents each comprises the common sequence or a complement thereof.

In any one or more of the embodiments disclosed herein, the kit comprises: reagents for allowing the capture agents to capture the target nucleic acids at locations on the substrate; reagents for generating an amplification product of each probe or probe set, wherein the amplification product comprises a plurality of stabilizing sequences complementary to the common sequence in the plurality of stabilization agents. In any one or more of the embodiments herein, the kit can comprise reagents for detecting signals associated with the amplification products of the plurality of probes or probe sets at locations on the substrate, thereby detecting the plurality of target nucleic acids at locations in the biological sample that correspond to the locations on the substrate.

In some aspects, provided herein is a kit for analyzing a biological sample, comprising: a substrate comprising a plurality of capture agents and a plurality of interspersing agents immobilized at interspersed locations on the substrate, wherein each capture agent comprises a capture domain capable of capturing a target nucleic acid of a plurality of target nucleic acids, and wherein interspersing agent is not capable of capturing a target nucleic acid of the plurality of target nucleic acids; and a plurality of probes or probe sets, wherein each probe or probe set binds to a target nucleic acid of the plurality of target nucleic acids or a complement of the target nucleic acid.

In any one or more of the embodiments disclosed herein, the kit comprises: reagents for allowing the capture agents to capture the target nucleic acids at locations on the substrate; reagents for generating an amplification product of each probe or probe set. In any one or more of the embodiments herein, the kit comprises reagents for detecting signals associated with the amplification products of the plurality of probes or probe sets at locations on the substrate, thereby detecting the plurality of target nucleic acids at locations in the biological sample that correspond to the locations on the substrate.

### BRIEF DESCRIPTION OF THE DRAWINGS

The drawings illustrate certain features and advantages of this disclosure. These embodiments are not intended to limit the scope of the appended claims in any manner.

FIG. 1 depicts an example work flow for detecting target nucleic acids using a substrate comprising capture agents and stabilization/interspersing agents. Analytes can be detected in the same round (e.g., Analytes 1 and 3) or in different rounds (e.g., Analytes 1 and 3 versus Analyte 2) of probe hybridization and probe complex or product generation and detection. In addition, probes and/or complexes or products thereof can be removed to allow the next round of probe hybridization and probe complex or product generation and detection. Each round may comprise one or more cycles of hybridization and detection of detectably labeled probes.

FIGS. 2A-2D depict an example work flow for detecting target RNAs using a substrate comprising capture agents and stabilization/interspersing agents.

FIG. 3 depicts an example of RCPs which are hybridized to stabilization agents and are spaced apart from each other by interspersing agents.

FIG. 4 is a schematic diagram depicting a sandwiching process between a first substrate comprising a biological sample (e.g., a tissue section on a slide) and a second substrate comprising a plurality of capture agents.

FIGS. 5A-5B show a perspective view of an example sample handling apparatus in a closed position (FIG. 5A) and in an open position (FIG. 5B), respectively.

FIG. 6A depicts the first angled over (superior to) the second substrate. FIG. 6B shows that as the first substrate lowers, and/or as the second substrate rises, the dropped side of the first substrate (e.g., a side of the slide angled toward the second substrate) may contact the drop of the reagent medium. FIG. 6C depicts a full closure of the sandwich between the first substrate and the second substrate with the spacer contacting both the first substrate and the second substrate and maintaining a separation distance and optionally the approximately parallel arrangement between the two substrates.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Biological sample (e.g., tissue sample) analysis encompassing spatial orientation, profiling and information regarding the position analytes are essential for many purposes, including understanding molecular basis of cell identity and development of diseases.

Methods such as those based on in situ rolling circle amplification (RCA) provide a high-throughput solution for detection of target nucleic acids in a biological sample. However, RCA-based methods performed within biological samples are associated with certain drawbacks. For instance, autofluorescence can be severe in some tissue types (such as FFPE tissues). Additionally, tissues are prone to detaching from the substrate, which could cause the signals to come out of focus during decoding of barcodes, which sometimes requires multiple cycles of probe hybridization, detection, and removal to optically resolve a large number of analytes using a limited number of fluorescent channels. In some aspects, the present disclosure provides methods and compositions for performing RCA outside of a biological sample while providing information regarding the location of the target nucleic acids at their positions in the biological sample.

In one aspect, provided herein is a method for analyzing a biological sample, comprising: a) contacting a biological sample (e.g., a tissue sample) with a substrate, wherein: the biological sample comprises a plurality of target nucleic acids, the substrate comprises a plurality of capture agents and a plurality of interspersing agents, wherein the capture agents and the interspersing agents are immobilized at interspersed positions on the substrate, and each capture agent comprises a capture domain capable of capturing a target nucleic acid of the plurality of target nucleic acids; b) providing conditions to allow the capture agents to capture the target nucleic acids, under which conditions the interspersing agents do not capture the target nucleic acids, thereby capturing a target nucleic acid by the capture agent immobilized at a location on the substrate, wherein the location corresponds to a location in the biological sample; c) contacting the substrate with a detectable probe that binds to the captured target nucleic acid or a product thereof at the location on the substrate; d) detecting a signal associated with the detectable probe or a complex or product thereof at the location on the substrate, thereby detecting the target nucleic acid at the corresponding location in the biological sample.

In one aspect, provided herein is a method for analyzing a biological sample, comprising: a) contacting a biological sample with a substrate, wherein: the biological sample comprises a plurality of target nucleic acids, the substrate comprises a plurality of capture agents and a plurality of stabilization agents, and each capture agent comprises a capture domain capable of capturing a target nucleic acid of the plurality of target nucleic acids; b) providing conditions to allow the capture agents to capture the target nucleic acids; c) contacting the substrate with a probe or probe set that binds to the captured target nucleic acid or a complement thereof immobilized at a location on the substrate; d) generating an amplification product associated with the probe or probe set, wherein the amplification product comprises a plurality of stabilizing sequences complementary to a sequence of the stabilization agents, thereby anchoring the amplification products at the location on the substrate; e) detecting a signal associated with the amplification product at the location on the substrate, thereby detecting the target nucleic acid at the corresponding location in the biological sample.

In some aspects, the presence of stabilization agents and/or interspersing agents on a uniform lawn of immobilized capture agents can provide RCPs with high positional stability (e.g., during probe hybridization and signal detection cycles) and mitigates autofluorescence observed in biological samples. Any one or more of the interspersing agents can function as a stabilization agent, for example, by hybridizing to an amplification product of a probe or probe set bound to a captured target nucleic acid or a complement thereof. In addition, any one or more of the stabilization agents function as an interspersing agent, for example, by spacing out adjacent capture agents such that on average, the physical separation of captured target nucleic acids on the substrate is increased compared to a reference substrate without interspersing agents interspersed among capture agents.

In some aspects, the capture of any one or more target nucleic acids can be targeted, for instance, by using target-specific capture domains. In some aspects, the capture of any one or more target nucleic acids can be non-targeted, for instance, by using capture domains that are not specific to any particular target nucleic acid. For instance, a poly(dT) sequence can be used as a common capture domain among the capture agents to capture different gene transcripts each having a poly(A) tail. In some embodiments, the common capture domain can be complementary to an overhang region (e.g., a 3′ overhang region) of a ligated probe generated from templated ligation of oligonucleotide probes hybridized to a target nucleic acid (e.g., target RNA) and the common capture domain can be any suitable sequence including a non-homopolymeric sequence. In some aspects, detection of the captured target nucleic acids or complements thereof on the substrate can be targeted, for instance, by using a probe or probe set targeting each specific target nucleic acid or a complement thereof, generating a complex or product of the target-specific probe or probe set, and detecting a signal associated with the complex or product of the target-specific probe or probe set.

In some aspects, one or more target nucleic acids can be transferred from a biological sample for capture by the capture agents on a substrate. The biological sample and the substrate can be aligned, for example, by using one or more fiducial markers on the substrate having the capture agents immobilized thereon, on the biological sample, and/or on a substrate having the biological sample immobilized thereon. In some embodiments, the methods comprise imaging the biological sample prior to transferring the target nucleic acids, and the imaging data can be overlaid with the detection data, such that the localizations of target nucleic acids in the originating sample are detected.

In some aspects, a lawn of immobilized capture agents and stabilization agents for RCPs can limit lateral diffusion of RCPs, preserving spatial information from the locations of the captured target nucleic acids on the substrate and corresponding locations of the target nucleic acids (prior to capture) in the biological sample.

In some aspects, a lawn of immobilized capture agents and interspersing agents can be used to control the density of various captured target nucleic acids on the substrate. For instance, in a reference substrate having a high density of immobilized capture agents, since each capture agent can capture a target nucleic acid, there can be multiple target nucleic acids (any two or more of which can be of the same sequence or different sequences) that are captured by capture agents in close vicinity to each other on the substrate. As such, when probes targeting the multiple target nucleic acids are hybridized and amplification products (e.g., RCPs) are generated on the substrate, detection of the amplification products using detectably labeled probes can lead to signal crowding. In some embodiments of the present disclosure, the presence of interspersing agents can be used to “dilute” the capture agents, and various dilutions can be used by adjusting the ratio of capture agents to interspersing agents. In some embodiments, the chances of multiple target nucleic acids being captured by capture agents in close vicinity to each other on the substrate can be reduced by using interspersing agents.

In some aspects, the methods disclosed herein can reduce autofluorescence and the risk of the biological sample detaching from the substrate by removing the biological sample before the RCA step and the subsequent detection. Location information can still be obtained even though RCA is not performed in situ in the biological sample. The biological sample can be imaged prior to digesting and/or removing the biological sample, and spatial locations of detected RCPs on the substrate can be correlated with positions in the biological sample (e.g., by overlaying data for the RCPs with data obtained from imaging the biological samples). In some aspects, RCPs generated on the substrate can be stabilized by the stabilization agents. In some aspects, since the capture agents can be spaced apart from one another by interspersing agents, the density of RCPs on the substrate can be reduced and the average distance between adjacent RCPs can be increased to allow better resolution of optical signals associated with the RCPs. In some embodiments, the optical signals associated with adjacent RCPs on the substrate are distinguishable. In some embodiments, the optical signals associated with adjacent RCPs on the substrate are resolvable using a fluorescence microscope. In some embodiments, the optical signals associated with adjacent RCPs on the substrate are decodable for analysis, e.g., using sequential hybridization of probes that directly or indirectly bind to the RCPs.

In some aspects, the methods disclosed herein comprise providing a substrate with a lawn of oligonucleotide molecules immobilized thereon, including capture agents and one or more other agents (e.g., stabilization agents and/or interspersing agents). In some embodiments, the biological sample is placed on the substrate. In some embodiments, the biological sample is placed on a first substrate, and a second substrate comprises a lawn of immobilized oligonucleotide molecules. In some aspects, the methods disclosed herein comprise permeabilizing the biological sample. Immobilized oligonucleotide molecules and methods for migrating and capturing target nucleic acids are described in greater detail in Sections II and III.

In some aspects, the methods disclosed herein comprise staining and imaging the biological sample. In some embodiments, the imaging data is analyzed in combination of detecting of rolling circle amplification products generated by RCA on substrate, thereby providing localization information of the target nucleic acids. Methods for staining and imaging biological samples and detecting rolling circle amplification products on the substrate are described in greater detail in Section III.

### II. Substrate and Immobilized Oligonucleotides

In some aspects, provided herein is a substrate comprising a plurality of oligonucleotide molecules immobilized on the substrate, including a plurality of capture agents each comprising a capture domain. In some embodiments, the capture domains of two or more of the plurality of capture agents comprise different nucleic acid sequences. In some embodiments, the capture domain of each of the plurality of capture agents is complementary to a different target nucleic acid sequence. In some embodiments, the capture domains of two or more of the plurality of capture agents comprise a common nucleic acid sequence. In some embodiments, the capture domain of each of the plurality of capture agents comprises the common nucleic acid sequence. In some embodiments, the capture domain of each of the plurality of capture agents comprises a poly(dT) sequence.

By hybridization of the capture domain to a target nucleic acid, an immobilized capture agent captures the target nucleic acid that is migrated out of the biological sample to the substrate. Once the double stranded complex between the capture domain and target nucleic acid is formed, the capture domain is then used as a primer for primer extension of the captured target nucleic acid to generate a complement of the captured target nucleic acid.

In some embodiments, the substrate comprises a lawn of oligonucleotide molecules immobilized thereon. In some cases, the immobilized oligonucleotides are not patterned on the substrate or capture area. In some embodiments, the substrate comprises i) a plurality of capture agents, and ii) a plurality of stabilization agents and/or a plurality of interspersing agents.

In some embodiments, the plurality of capture agents, the plurality of stabilization agents, and/or the plurality of interspersing agents are directly or indirectly immobilized on the substrate. In some embodiments, the plurality of capture agents, the plurality of stabilization agents, and/or the plurality of interspersing agents are covalently or non-covalently immobilized on the substrate. In some embodiments, the plurality of capture agents, the plurality of stabilization agents, and/or the plurality of interspersing agents are randomly distributed on the substrate.

In some embodiments, the plurality of capture agents, the plurality of stabilization agents, and/or the plurality of interspersing agents form an oligonucleotide lawn on the substrate. In some embodiments, the plurality of capture agents, the plurality of stabilization agents, and/or the plurality of interspersing agents are not distributed in a pattern of discrete features on the substrate. In some embodiments, the plurality of stabilization agents and/or the plurality of interspersing agents can be interspersed among the plurality of capture agents which form a lawn of oligonucleotides uniformly distributed on the substrate. In some embodiments, the plurality of capture agents are interspersed among the plurality of stabilization agents which form a lawn of oligonucleotides uniformly distributed on the substrate. In some embodiments, the plurality of capture agents can be interspersed among the plurality of interspersing agents which form a lawn of oligonucleotides uniformly distributed on the substrate.

In some embodiments, the ratio between molecules of the plurality of capture agents and molecules of the plurality of stabilization and/or interspersing agents on the substrate or in a capture area thereof is greater than 100:1, about 100:1, about 50:1, about 20:1, about 10:1, about 5:1, about 2:1, about 1:1, about 1:2, about 1:5, about 1:10, about 1:20, about 1:50, about 1:100, or less than 1:100. In some embodiments, prior to immobilization of the plurality of capture agents and the plurality of stabilization and/or interspersing agents on the substrate, the method comprises mixing molecules of the plurality of capture agents and molecules of the plurality of stabilization and/or interspersing agents at a ratio of greater than 100:1, about 100:1, about 50:1, about 20:1, about 10:1, about 5:1, about 2:1, about 1:1, about 1:2, about 1:5, about 1:10, about 1:20, about 1:50, about 1:100, or smaller than 1:100.

In some embodiments, the density of molecules of the plurality of capture agents and molecules of the plurality of stabilization and/or interspersing agents on the substrate or in a capture area on the substrate can be between about 0.1 and about 100 picomoles per 10 μm² area. In some embodiments, the density of molecules of the plurality of capture agents and molecules of the plurality of stabilization and/or interspersing agents on the substrate or in a capture area on the substrate can be between about 1 and about 10 picomoles per 10 μm² area. In some embodiments, the density of molecules of the plurality of capture agents and molecules of the plurality of stabilization and/or interspersing agents on the substrate or in a capture area on the substrate can be about 1, about 2, about 3, about 4, about 5, about 6, about 7, about 8, about 9, or about 10 picomoles per 10 μm² area.

In some embodiments, the method does not comprise arranging molecules of the plurality of capture agents and/or molecules of the plurality of stabilization and/or interspersing agents in distinctive features on the substrate. In some embodiments, the method does not comprise printing molecules of the plurality of capture agents and/or molecules of the plurality of stabilization and/or interspersing agents in array spots on the substrate.

In some embodiments, molecules of the plurality of capture agents and/or molecules of the plurality of stabilization and/or interspersing agents can be uniformly distributed on the substrate or in a capture area on the substrate. In some embodiments, one or more molecules of the plurality of capture agents and/or the plurality of stabilization and/or interspersing agents can each comprise a spatial barcode corresponding to the location of the capture agent or stabilization and/or interspersing agent on the substrate. In some embodiments, none of the plurality of capture agents and the plurality of stabilization and/or interspersing agents comprises a spatial barcode corresponding to the location of the capture agent or stabilization and/or interspersing agent on the substrate.

In some embodiments, the capture domains of two or more of the plurality of capture agents comprise different nucleic acid sequences. In some embodiments, the capture domain of each of the plurality of capture agents is complementary to a different target nucleic acid sequence, such as a transcript of a different gene. In some embodiments, the capture domains of two or more of the plurality of capture agents comprises a common nucleic acid sequence. In some embodiments, the capture domain of each of the plurality of capture agents comprises the common nucleic acid sequence. In some embodiments, the capture domain of each of the plurality of capture agents comprises a poly(dT) sequence. In some embodiments, the poly(dT) sequence can be about 10, about 15, about 20, about 25, about 30, or more than 30 nucleotides in length. In some embodiments, each of the plurality of target nucleic acids can comprise a poly(A) sequence. In some embodiments, the plurality of target nucleic acids can comprise mRNA of different genes.

In some embodiments, two or more of the plurality of stabilization and/or interspersing agents comprise different nucleic acid sequences. In some embodiments, two or more of the plurality of stabilization and/or interspersing agents comprise a common nucleic acid sequence. In some embodiments, each of the plurality of stabilization and/or interspersing agents comprise the common nucleic acid sequence. In some embodiments, the common nucleic acid sequence in the stabilization agents are complementary to the plurality of stabilizing sequences in the amplification product. In some embodiments, the common nucleic acid sequence in the interspersing agents is not complementary to any of the target nucleic acids. In some embodiments, the common nucleic acid sequence in the interspersing agents is not complementary to a sequence in the probe complexes or products. In some embodiments, the common nucleic acid sequence in the interspersing agents can but does not need to be complementary to a sequence in the amplification products (e.g., the RCPs).

In some embodiments, the plurality of stabilization and/or interspersing agents do not comprise a capture domain capable of capturing a target nucleic acid of the plurality of target nucleic acids. In some embodiments, the plurality of stabilization and/or interspersing agents do not comprise a sequence complementary to a target nucleic acid of the plurality of target nucleic acids. In some embodiments, the plurality of stabilization and/or interspersing agents do not comprise a sequence of more than 10, 20, or 30 consecutive nucleotides complementary to a target nucleic acid of the plurality of target nucleic acids. In some embodiments, the plurality of stabilization and/or interspersing agents do not comprise a poly(dT) sequence of about 10, about 15, about 20, about 25, about 30, or more than 30 nucleotides in length. In some embodiments, the plurality of stabilization and/or interspersing agents do not comprise a poly(dT) sequence.

Oligonucleotides may be attached to the substrate according to the methods set forth in U.S. Pat. Nos. 6,737,236, 7,259,258, 7,375,234, 7,427,678, 5,610,287, 5,807,522, 5,837,860, and 5,472,881; U.S. Patent Application Publication Nos. 2008/0280773 and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chriscy et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392. The entire contents of each of the foregoing documents are incorporated herein by reference.

The oligonucleotide molecules can be attached to a substrate or capture area thereon using a variety of techniques. In some embodiments, the oligonucleotide molecules are immobilized to a substrate by chemical immobilization. For example, a chemical immobilization can take place between functional groups on the substrate and corresponding functional elements on the oligonucleotide molecules. Examples of corresponding functional elements in the oligonucleotide molecules can either be an inherent chemical group of the oligonucleotide molecule, e.g., a hydroxyl group, or a functional element can be introduced on to the oligonucleotide molecule. An example of a functional group on the substrate is an amine group or an N-hydroxysuccinimide (NHS) ester. In some embodiments, the oligonucleotide molecule to be immobilized can comprise one or more amine groups (such as —NH₂ groups) at a 5′ region, which can react with a functional group such as NHS on the substrate. In some embodiments, the oligonucleotide molecule to be immobilized includes a functional amine group or is chemically modified in order to include a functional amine group. In some embodiments, the substrate comprising the oligonucleotide molecules is manufactured by dipping the substrate (e.g., the substrate comprising a functional group coating) in an oligonucleotide solution (e.g., a functionalized oligonucleotide solution). In some embodiments, the oligonucleotide molecules are uniformly distributed on the substrate. In some embodiments, the oligonucleotide molecules are uniformly distributed on a capture area of the substrate. In some embodiments, the oligonucleotides completely cover the capture area on the substrate. In some embodiments, functionalized oligonucleotide molecules are immobilized on a functionalized substrate using covalent methods. Methods for covalent attachment include, for example, condensation of amines and activated carboxylic esters (e.g., N-hydroxysuccinimide esters); condensation of amine and aldehydes under reductive amination conditions; and cycloaddition reactions such as the Diels-Alder [4+2] reaction. 1,3-dipolar cycloaddition reactions, and [2+2] cycloaddition reactions. Methods for covalent attachment also include, for example, click chemistry reactions, including [3+2] cycloaddition reactions (e.g., Huisgen 1,3-dipolar cycloaddition reaction and copper(I)-catalyzed azide-alkyne cycloaddition (CuAAC)); thiol-ene reactions; the Diels-Alder reaction and inverse electron demand Diels-Alder reaction; [4+1] cycloaddition of isonitriles and tetrazines; and nucleophilic ring-opening of small carbocycles (e.g., epoxide opening with amino oligonucleotides). Methods for covalent attachment also include, for example, maleimides and thiols; and para-nitrophenyl ester-functionalized oligonucleotides and polylysine-functionalized substrate. Methods for covalent attachment also include, for example, disulfide reactions; radical reactions (see, e.g., U.S. Pat. No. 5,919,626, the entire contents of which are herein incorporated by reference); and hydrazide-functionalized substrate (e.g., wherein the hydrazide functional group is directly or indirectly attached to the substrate) and aldehyde-functionalized oligonucleotides (see, e.g., Yershov et al. (1996) Proc. Natl. Acad. Sci. USA 93, 4913-4918, the entire contents of which are herein incorporated by reference). In some embodiments, the substrate comprising the oligonucleotide molecules is manufactured by dipping the substrate (e.g., the substrate comprising a functional group coating) in an oligonucleotide solution (e.g., a functionalized oligonucleotide solution).

In some embodiments, functionalized oligonucleotide molecules are immobilized on a functionalized substrate using photochemical covalent methods. Methods for photochemical covalent attachment include, for example, immobilization of antraquinone-conjugated oligonucleotides (see, e.g., Koch et al. (2000) Bioconjugate Chem. 11, 474-483, the entire contents of which are herein incorporated by reference). In some embodiments, the substrate comprising the oligonucleotide molecules is manufactured by dipping the substrate (e.g., the substrate comprising a functional group coating) in an oligonucleotide solution (e.g., a functionalized oligonucleotide solution).

In some embodiments, functionalized oligonucleotide molecules are immobilized on a functionalized substrate using non-covalent methods. Methods for non-covalent attachment include, for example, biotin-functionalized oligonucleotides and streptavidin-treated substrates (see, e.g., Holmstrom et al. (1993) Analytical Biochemistry 209, 278-283 and Gilles et al. (1999) Nature Biotechnology 17, 365-370, the entire contents of which are herein incorporated by reference). In some embodiments, the substrate comprising the oligonucleotide molecules is manufactured by dipping the substrate (e.g., the substrate comprising a functional group coating) in an oligonucleotide solution (e.g., a functionalized oligonucleotide solution).

In any of the embodiments herein, the 3′ terminal nucleotides of the immobilized oligonucleotide molecules (e.g., capture agents, stabilization agents, and/or interspersing agents) can be distal to the substrate. In any of the embodiments herein, the 5′ terminal nucleotides of the immobilized oligonucleotide molecules can be more proximal to the substrate than the 3′ terminal nucleotides. In any of the embodiments herein, one or more nucleotides at or near the 5′ terminus of each immobilized oligonucleotide can be directly or indirectly attached to the substrate, thereby immobilizing the oligonucleotides. In any of the embodiments herein, the 3′ terminus of each immobilized oligonucleotide is the end that is not immobilized on the substrate. In any of the embodiments herein, the oligonucleotide molecules are immobilized on the substrate via a 5′ amino group. In any of the embodiments herein, the oligonucleotide molecules comprise a free 3′ hydroxyl group.

In some embodiments, the capture agents comprise a capture domain. In some embodiments, the capture domain is common to the capture agents. In some embodiments, the capture domain is used to capture target nucleic acids. In some embodiments, the capture domain is at the 3′ terminus of the capture agents.

In some embodiments, the capture domain comprises at least 10 nucleotides. In some embodiments, the capture domain comprises 10-40 nucleotides, In some embodiments, the capture domain comprises 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 30 nucleotides, 35 nucleotides, or 40 nucleotides.

In some aspects, the length of the capture domain relates to the hybridization condition and the preferred melting temperature (Tₘ) of the sequence. Several equations for calculating the Tₘ can be used, for instance, a simple estimate of the Tₘ value may be calculated by the equation, Tₘ=81.5+0.41 (% G+C), when a nucleic acid is in aqueous solution at 1 M NaCl (see, e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ. In some embodiments, the Tₘ is between any of about 30° C. and about 90° C., about 30° C. and about 80° C., about 50° C. and about 80° C., about 50° C. and about 70° C., about 30° C. and about 32° C., about 32° C. and about 34° C., about 34° C. and about 36° C., about 36° C. and about 38° C., or about 38° C. and about 40° C. In some embodiments, the Tₘ is optimized based on various conditions, such as the concentrations of DMSO, formamide, salts, and/or primers.

In some embodiments, the immobilized oligonucleotide molecules (e.g., capture agents, stabilization agents, and/or interspersing agents) comprise linkers (e.g., linkers between the capture domain and the substrate). In some embodiments, the linker is a nucleic acid sequence of any one of about 10, about 20, about 30, about 40, about 50, or about 60 nucleotides in length, or any range between any of said values. In some cases, the linker is a nucleic acid sequence of between about 5 and about 20, between about 5 and about 30, between about 5 and about 40, between about 10 and about 25, between about 10 and about 35, between about 10 and about 50, between about 10 and about 60, or between about 10 and about 100 nucleotides in length.

In some embodiments, the linker is an organic linker. Examples of organic linkers include but are not limited to poly-glycols. In some instances, the linker is an alkyl linker or an oligoethylene glycol linker. In some cases, the linker is a tetraethylene glycol, hexaethylene glycol, or decaethylene glycol linker. In some embodiments, the linker is a hydrophilic linker.

In some cases, the linker is a cleavable linker. In some embodiments, the linker is a photocleavable linker, a UV-cleavable linker, an enzyme-cleavable linker, or a pH-sensitive cleavable linker.

In some embodiments, the immobilized oligonucleotides (e.g., capture agents, stabilization agents, and/or interspersing agents) comprise one or more endonuclease-resistant linkages (e.g., any non-natural nuclease resistant internucleoside linkages, such as phosphoramadite linkages). In some embodiments, the immobilized oligonucleotides comprise one, two, three, or more phosphoramadite linkages. In some cases, the one, two, three, or more phosphoramadite linkages are at the 3′ end. In some embodiments, the endonuclease-resistant linkages improve stability of the oligonucleotide molecules immobilized on the substrate.

In some embodiments, a substrate herein can be any support that is insoluble in aqueous liquid and which allows for positioning of biological samples, immobilized oligonucleotide molecules (e.g., capture agents, stabilization agents, and/or interspersing agents), reagents (e.g., probes) and/or amplification products (e.g., rolling circle amplification products) on the support.

In some cases, a first substrate and a second substrate are provided, wherein the first substrate comprises the oligonucleotide molecules (e.g., capture agents, stabilization agents, and/or interspersing agents) immobilized thereon and the biological sample is on the second substrate. In some embodiments, target nucleic acids are migrated from the biological sample to the first substrate where they are captured, reverse transcribed (in cases where the target nucleic acids are RNA). In some cases, the reverse transcribed target nucleic acids are amplified. In other cases, the biological sample is on the same substrate as the immobilized oligonucleotide molecules (e.g., capture agents, stabilization agents, and/or interspersing agents).

In some embodiments, the biological sample is attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

In some aspects, a substrate functions as a support for direct or indirect attachment of oligonucleotides (e.g., to form a lawn of immobilized oligonucleotide molecules). In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and which allows for positioning of biological samples, immobilized oligonucleotide molecules, and/or rolling circle amplification products generated on the substrate.

A wide variety of different substrates can be used, as long as the substrate is compatible with the sample and sample processing, the reagents and reactions, and signal detection (e.g., optical imaging such as fluorescence microscopy). A substrate can be any suitable support material. For instance, the first substrate and/or the second substrate can be any solid or semi-solid support upon which a biological sample can be mounted. The first substrate and/or the second substrate can include, but are not limited to, glass, modified and/or functionalized glass, hydrogels, films, membranes, plastics, paper, nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene, polycarbonate, and polymer monoliths. In some embodiments, the first substrate and/or the second substrate comprises an inert material or matrix (e.g., glass slides) that has been functionalized by, for example, treating the substrate with a material comprising reactive groups which facilitate mounting of the biological sample. The first substrate and/or the second substrate comprises a substantially flat planar surface. The first substrate and/or the second substrate can be a slide, e.g., a glass slide. For example, a glass slide such as a cover slip may be used. The first substrate and/or the second substrate can be transparent. The first substrate and/or the second substrate can also correspond to or be part of a flow cell.

In some instances, a substrate herein (e.g., a first or second substrate) is between about 0.01 mm and about 5 mm, e.g., between about 0.05 mm and about 3 mm, between about 0.1 mm and about 2.5 mm, between about 0.2 mm and about 2 mm, between about 0.5 mm and about 1.5 mm, or about 1 mm in thickness. In some embodiments, the substrate (e.g., a first substrate or a second substrate) is or is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm in thickness, or of a thickness in between any of the aforementioned values.

The substrate can also correspond to a flow cell. Flow cells can be formed of any of the foregoing materials, and can include channels that permit reagents, solvents, and molecules to pass through the flow cell.

Among the examples of substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In some embodiments, a substrate is coated with a surface treatment such as poly(L)-lysine. In some embodiments, a substrate surface is modified by silanization. In some embodiments, a substrate surface is modified by applying epoxy-silane or amino-silane. In some embodiments, the substrate is treated by silanation, e.g., with epoxy-silane, amino-silane, and/or by a treatment with polyacrylamide.

The substrate can generally have any suitable form or format. For example, in some embodiments the substrate is flat, curved, e.g., convexly or concavely curved towards the area where the interaction between a biological sample, e.g., tissue sample, and the substrate takes place. In some embodiments, the substrate is a flat, e.g., planar, chip or slide. In some embodiments, the substrate contains one or more patterned surfaces within the substrate (e.g., channels, wells, projections, ridges, divots, etc.).

A substrate can be of any desired shape. For example, a substrate in some embodiments is typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure is any appropriate type of support having a flat surface (e.g., a chip or a slide such as a microscope slide).

In some embodiments, a substrate includes one or more markings on a surface of the substrate, e.g., to provide guidance for correlating spatial information (e.g., cell boundaries or histology from staining and/or immunohistochemistry) with detected rolling circle amplification products on the substrate. For example, in some embodiments a substrate is marked with a grid of lines (e.g., to allow the size of objects seen under magnification to be easily estimated and/or to provide reference areas for counting objects). In some embodiments, fiducial markers are included on the substrate. In some embodiments, such markings are made using techniques including, but not limited to, printing, sand-blasting, and depositing on the surface.

In some instances, the second substrate having a sample attached thereto does not comprise immobilized oligonucleotide molecules on the second substrate. Instead, the immobilized oligonucleotide molecules comprising capture domains are provided on one or more first substrates, to which the biological sample is introduced. In some instances, the biological sample is introduced after imaging the biological sample. For example, a second substrate comprising a biological sample (which may but does not need to have previously undergone an in situ assay involving imaging the biological sample), and a first substrate, e.g., comprising a lawn of immobilized oligonucleotide molecules, are subjected to a sandwiching process described herein to facilitate molecular interaction and/or transfer of target nucleic acid from the sample to the first substrate.

In some embodiments, the first substrate and/or the second substrate include one or more markings on its surface, e.g., to provide guidance for aligning at least a portion of the biological sample with a plurality of capture agents on the first substrate during a sandwich process disclosed herein (e.g., in Section III.A.). For example, the first substrate and/or the second substrate can include a sample area indicator identifying the sample area. In some embodiments, during a sandwiching process described herein, the sample area indicator on the second substrate is aligned with an area of the first substrate comprising a plurality of capture agents. In some embodiments, the first and/or second substrate include a fiducial mark. In some embodiments, the first and/or second substrate does not comprise a fiducial mark. In some embodiments, the first substrate does not comprise a fiducial mark and the second substrate comprises a fiducial mark. In some embodiments, such markings are made using techniques including, but not limited to, printing, sand-blasting, and depositing on the surface.

In some embodiments, imaging is performed using one or more fiducial markers, e.g., objects placed in the field of view of an imaging system which appear in the image produced. Fiducial markers are typically used as a point of reference or measurement scale. Fiducial markers and their uses are described in further detail in, e.g., WO 2020/176788 A1, the entire contents of which are incorporated herein by reference.

In some embodiments, the biological sample on the second substrate is stained using any of the methods described herein. In some instances, the biological sample is imaged, capturing the stain pattern created during the stain step. In some instances, the biological sample then is destained prior to the sandwiching process (e.g., migration described in Section III).

To facilitate visualization, in some embodiments, biological samples are stained using a wide variety of stains and staining techniques. In some embodiments, for example, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample are segmented using one or more images taken of the stained sample.

In some embodiments, the stain is performed using a lipophilic dye. In some examples, the staining is performed with a lipophilic carbocyanine or aminostyryl dye, or analogs thereof (e.g., DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, Coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

In some embodiments, the sample is stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, and Giemsa stain.

In some embodiments, the biological sample is stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes biological staining using hematoxylin. In some embodiments, staining includes identifying analytes using fluorescently-conjugated antibodies, e.g., by immunofluorescence. In some embodiments, a biological sample is stained using two or more different types of stains, or two or more different staining techniques. For example, in some embodiments, a biological sample is prepared by staining and imaging using one technique (e.g., H&E staining and brightfield imaging), followed by staining and imaging using another technique (e.g., IHC/IF staining and fluorescence microscopy) for the same biological sample. In some instances, a biological sample is stained.

In some instances, methods for immunofluorescence include a blocking step. The blocking step can include the use of blocking probes to decrease unspecific binding of the antibodies. In some instances, the blocking step can further include contacting the biological sample with a detergent. In some instances, the detergent can include Triton X-100™. The method can further include an antibody incubation step. In some embodiments, the antibody incubation step effects selective binding of the antibody to antigens of interest in the biological sample. In some embodiments, the antibody is conjugated to an oligonucleotide (e.g., an oligonucleotide-antibody conjugate as described herein). In some embodiments, the antibody is not conjugated to an oligonucleotide. In some embodiments, the method further comprises an antibody staining step. The antibody staining step can include a direct method of immunostaining in which a labelled antibody binds directly to the analyte being stained for. Alternatively, the antibody staining step can include an indirect method of immunostaining in which a first antibody binds to the analyte being stained for, and a second, labelled antibody binds to the first antibody. In some embodiments, the antibody staining step is performed prior to sandwich assembly. In some embodiments wherein an oligonucleotide-antibody conjugate is used in the antibody incubation step, the method does not comprise an antibody staining step.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, image are captured using high resolution techniques (e.g., having 300 dots per square inch (dpi) or greater). For example, in some embodiments images are captured using brightfield imaging (e.g., in the setting of hematoxylin or H&E stain), or using fluorescence microscopy to detect adhered labels. In some instances, high resolution images are captured temporally using e.g., confocal microscopy. In some instances, a low resolution image is captured. In some embodiments, a low resolution image (e.g., images that are about 72 dpi and normally have an RGB color setting) is captured at any point of the workflow, including but not limited to staining, destaining, permeabilization, sandwich assembly, and migration of the analytes. In some instances, a low resolution image is taken during permeabilization of the biological sample.

In some embodiments, the location of the one or more additional analytes in a biological sample are determined by immunofluorescence. In some embodiments, one or more detectable labels (e.g., fluorophore-labeled antibodies, nucleic acid probes disclosed herein) bind to the one or more analytes that are captured (hybridized to) by a probe on the first slide and the location of the one or more analytes is determined by detecting the labels under suitable conditions. In some embodiments, one or more fluorophore-labeled antibodies are used to conjugate to a moiety that associates with a probe on the first slide or the analyte that is hybridized to the probe on the first slide. In some instances, the location(s) of the one or more analytes is determined by imaging the fluorophore-labeled antibodies when the fluorophores are excited by a light of a suitable wavelength. In some embodiments, the location of the one or more analytes in the biological sample is determined by correlating the immunofluorescence data to an image of the biological sample. In some instances, the tissue is imaged throughout the permeabilization step.

In some instances, the biological samples are destained. In some instances, destaining occurs prior to permeabilization of the biological sample. By way of example only, in some embodiments, H&E staining is destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

### III. Methods

### A. Target Nucleic Acid Migration and Capture

In some aspects, the methods provided herein comprise capturing target nucleic acids using immobilized oligonucleotide molecules on a substrate and using the immobilized oligonucleotide molecules as primers to perform primer extension of the capturing target nucleic acids, thereby providing a plurality of complements (e.g., cDNA) of the capture target nucleic acids on the substrate. In some embodiments, the complements (e.g., cDNA) are recognized by probes or probe sets for on-substrate rolling circle amplification of circularized probes (e.g., in some instances a circularized probe is generated from a probe or probe set hybridized to a captured target nucleic acid or a complement thereof) at positions on the substrate. In some embodiments, the on-substrate rolling circle amplification provides a number of advantages, including increased rolling circle amplification efficiency and sensitivity, positional stability of rolling circle amplification products immobilized on the substrate, and/or reduced autofluorescence by detecting amplification products on a substrate rather than in the biological sample.

In some aspects, the target nucleic acids are captured when contacting the biological sample comprising the target nucleic acids with a substrate including immobilized oligonucleotide molecules comprising capture domains (e.g., a substrate with immobilized oligonucleotide molecules forming a lawn on a capture area of the substrate). As used herein, “contact,” “contacted,” and/or “contacting,” a biological sample with a substrate refers to any contact (e.g., direct or indirect) such that the immobilized oligonucleotide molecules can interact (e.g., bind covalently or non-covalently (e.g., hybridize)) with nucleic acids (e.g., the target nucleic acids) from the biological sample. In some embodiments, capture is achieved actively (e.g., using electrophoresis) or passively (e.g., using diffusion).

In some embodiments, the plurality of target nucleic acids can comprise a cellular RNA, a cDNA, a genomic DNA, and/or a reporter oligonucleotide. In some embodiments, the reporter oligonucleotide is conjugated to a binding moiety that binds to a non-nucleic acid analyte. In some embodiments, the binding moiety comprises an antibody or epitope binding fragment thereof. In some embodiments, the plurality of target nucleic acids do not comprise a ligated circularizable probe generated using a cellular nucleic acid (e.g., cellular RNA or genomic DNA) as a template in a biological sample. In some embodiments, the plurality of target nucleic acids do not comprise a circularized probe generated in a biological sample. In some embodiments, the plurality of target nucleic acids do not comprise a circularized probe generated at locations in a cell or tissue sample.

In some embodiments, the plurality of target nucleic acids comprises a linear ligated probe generated using a nucleic acid (e.g., cellular RNA, cDNA, or genomic DNA) as a template in a biological sample. In some embodiments, the linear ligated probe is generated at locations in a cell or tissue sample, for instance, using RNA molecules at the locations as ligation templates for ligating two or more probe parts. In some embodiments, the linear ligated probe is generated by ligating a first probe comprising a first target hybridizing region and a second probe comprising a second target hybridizing region, where the first and second target hybridizing regions are ligated using a target nucleic acid as template. The linear ligated probe can comprise a 3′ overhang and/or a 5′ overhang, either of which may be captured by a capture agent disclosed herein and/or comprise a barcode region. In some embodiments, the captured linear ligated probe is used as template for an extension reaction to generate an extension product comprising one or more sequences of the linear ligated probe. A probe or probe set disclosed herein (e.g., a circularizable probe) can bind to the extension product.

In some embodiments, the conditions provided to allow the capture agents to capture the target nucleic acids comprise releasing the target nucleic acids from the biological sample. In some embodiments, the method comprises digesting or permeabilizing the biological sample. In some cases, the biological sample is digested using a proteinase (e.g., Proteinase K). In some embodiments, the conditions provided to allow the capture agents to capture the target nucleic acids comprise migrating the target nucleic acids towards the substrate. In some embodiments, the conditions provided to allow the capture agents to capture the target nucleic acids comprise contacting the target nucleic acids with the capture agents and the stabilization agents, such that target nucleic acids captured at adjacent locations are spaced from one another by one or more stabilization agents.

In some aspects, the target nucleic acids are migrated toward the substrate comprising the immobilized oligonucleotide molecules. In some embodiments, the target nucleic acids are migrated along an axis substantially perpendicular to the substrate, preserving the original spatial localization of the target nucleic acids in two-dimensional space. In some embodiments, the biological sample is on the substrate comprising a lawn of immobilized oligonucleotide molecules as described above. In some embodiments, the biological sample is digested to facilitate migration of the target nucleic acids. In some embodiments, at least a portion of the target nucleic acids in a biological sample are captured by hybridization to the capture domains of a plurality of the immobilized oligonucleotide molecules.

In some embodiments, the biological sample is on a second substrate and the substrate comprising a lawn of immobilized oligonucleotide molecules is a first substrate. In some cases, the target nucleic acids are migrated from the biological sample toward the substrate. In some embodiments, at least a portion of the target nucleic acids are captured by hybridization to the capture domains of a plurality of the immobilized oligonucleotide molecules.

In some embodiments, the biological sample is on a second substrate, and the method comprises migrating the target nucleic acids toward a first substrate comprising a lawn of immobilized oligonucleotide molecules. In some embodiments, the target nucleic acids are transferred to the first substrate without the biological sample, and captured by the oligonucleotide molecules. In some embodiments, the transfer of the target nucleic acids from the sample on the second substrate to the oligonucleotide molecules on the first substrate is facilitated by a sandwiching process.

In some embodiments, a biological sample is provided (e.g., placed) on a substrate comprising a lawn of immobilized oligonucleotide molecules. In some embodiments, a biological sample is provided (e.g., placed) on a second substrate and a first substrate is provided comprising a lawn of immobilized oligonucleotide molecules.

In some embodiments, the methods disclosed herein comprises contacting the biological sample with a reagent medium. In some embodiments, the reagent medium comprises a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., Proteinase K). In some embodiments, the detergent is an anionic detergent (e.g., SDS or N-lauroylsarcosine sodium salt solution). Example permeabilization reagents are described in US. Patent Application Pub. No. 20210189475, which is incorporated by reference in its entirety.

A reagent medium may be applied between the first substrate and the second substrate and create a liquid interface between the two substrates. The reagent medium may be a permeabilization solution which permeabilizes and/or digests the sample. In some embodiments wherein the sample has been pre-permeabilized, the reagent medium is not a permeabilization solution. In some embodiments, target nucleic acids in the biological sample released from the biological sample, actively or passively migrate (e.g., diffuse) toward the lawn of oligonucleotide molecules. In some embodiments, the active migration is via electrophoresis. Electrophoretic migration methods are further described in US. Patent Application Pub. No. 20210189475, which is hereby incorporated by reference.

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Example lysis reagents are described in US. Patent Application Pub. No. 20210189475, which is incorporated by reference in its entirety.

In some embodiments, the reagent medium comprises a protease. Example proteases include, e.g., pepsin, trypsin, pepsin, elastase, and Proteinase K. Example proteases are described in US. Patent Application Pub. No. 20210189475, which is incorporated by reference in its entirety.

In some embodiments, the reagent medium comprises a detergent. Example detergents include sodium dodecyl sulfate (SDS), sarkosyl, saponin, Triton X-100™, and Tween-20™. Example detergents are described in US. Patent Application Pub. No. 20210189475, which is incorporated by reference in its entirety.

In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS), Proteinase K, pepsin, N-lauroylsarcosine, RNase, and a sodium salt thereof.

In some embodiments, a fixed tissue sample mounted on a second substrate (e.g., a slide-mounted tissue sample) is decrosslinked. In some embodiments, the sample is washed (e.g., with a buffer). In some embodiments, the target nucleic acids are released from the tissue under sandwich conditions as described herein. For the sandwich conditions, in some embodiments the tissue-mounted slide is aligned with a first substrate comprising a lawn of immobilized capture oligonucleotide molecules and permeabilized with a reagent medium in the sandwich configuration as described herein. In some embodiments, the reagent medium comprises a permeabilization agent (e.g., Proteinase K). After capture of the target nucleic acids using the capture domain of the immobilized oligonucleotide molecules, in some embodiments the tissue slide is removed (e.g., in some embodiments the sandwich is “opened” or “broken”).

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In some embodiments, the reagent medium includes a wetting agent.

In some instances, the biological sample is in contact with the reagent medium for about 1 minute. In some instances, the biological sample is in contact with the reagent medium for about 5 minutes. In some instances, the biological sample is in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the biological sample is in contact with the reagent medium for about 1-60 minutes, about 10-60 minutes, about 30-60 minutes, or about 20-45 minutes. In some instances, the biological sample is in contact with the reagent medium for about 30 minutes. In some instances, the biological sample is in contact with the reagent medium for at least about any of 30, 40, 45, 50, 55, or 60 minutes. In some instances, the biological sample is in contact with the reagent medium for about 60 minutes.

Example substrates similar to the first substrate (e.g., a substrate having no immobilized oligonucleotide molecules for probe capture) and/or the second substrate are described in Section II above. In some embodiments, the sandwiching process may be facilitated by a device, sample holder, sample handling apparatus, or system described in, e.g., US. Patent Application Pub. No. 20210189475, WO 2021/252747, or WO 2022/061152, which are incorporated herein by reference.

In some instances, provided herein is a sandwiching process between a second substrate comprising a biological sample (e.g., a tissue section on a slide) and a first substrate comprising a lawn of oligonucleotide molecules. For example, the first substrate is aligned with the second substrate, such that at least a portion of the biological sample is aligned with at least a portion of the lawn (e.g., aligned in a sandwich configuration). As shown, the first substrate (e.g., capture agent slide) may be positioned superior to the second substrate (e.g., sample slide). In some embodiments, the second substrate (e.g., sample slide) is in a superior position to the first substrate (e.g., capture agent slide). In some embodiments, the biological sample on the second substrate does not come into direct contact with the first substrate (e.g., using one or more spacers). When the first and second substrates are aligned, one or more target nucleic acids are released from the biological sample on the second substrate and actively or passively migrate to the first substrate for capture. In some embodiments, the migration occurs while the aligned portions of the biological sample and the oligonucleotide lawn are contacted with a reagent that permeabilizes the biological sample, e.g. any permeabilization reagent disclosed herein. In some embodiments, the permeabilization reagent is Proteinase K. The released one or more target nucleic acids may actively or passively migrate towards the substrate and be captured by the lawn of oligonucleotide molecules comprising capture agents as well as stabilization and/or interspersing agents.

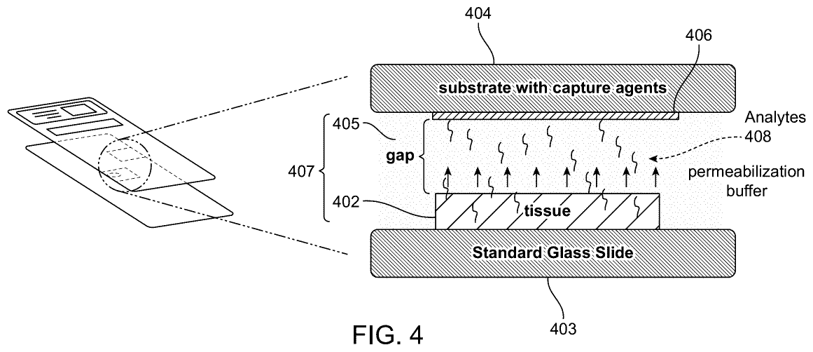

FIG. 4 is a schematic diagram depicting a sandwiching process between a substrate comprising a biological sample (e.g., a tissue section 402 on a slide 403) and a substrate comprising a plurality of capture agents. In some embodiments, the substrate comprising a plurality of capture agents is e.g., a slide 404 that is populated with capture agents 406. In some embodiments, capture agents 406 are spatially barcoded. In some embodiments, capture agents 406 do not comprise spatial barcodes. During the example sandwiching process, the first substrate is aligned with the second substrate, such that at least a portion of the biological sample is aligned with at least a portion of the capture agents (e.g., aligned in a sandwich configuration). As shown, the substrate with the capture agents (e.g., slide 404) is in a superior position to the substrate with the tissue (e.g., slide 403). In some embodiments, the substrate with the tissue (e.g., slide 403) may be positioned superior to the substrate with the capture agents (e.g., slide 404). A reagent medium 405 within a gap 407 between substrates (e.g., slides) creates a liquid interface between the two substrates. The reagent medium may be a permeabilization solution which permeabilizes and/or digests the sample 402. In some embodiments wherein the sample 402 has been pre-permeabilized, the reagent medium is not a permeabilization solution. In some embodiments, analytes (e.g., target nucleic acids) 408 of the biological sample 402 may release from the biological sample, actively or passively migrate (e.g., diffuse) across the gap 407 toward the capture agents 406, and bind on the capture agents 406. In some embodiments, the active migration is via electrophoresis. Electrophoretic migration methods are further described in US. Patent Application Pub. No. 20210189475, which is hereby incorporated by reference.

In some embodiments, an extension reaction may be performed to extend the capture agents 406 using captured target nucleic acids as template. In some embodiments, the extension reaction is performed separately from the sample handling apparatus described herein that is configured to perform the example sandwiching process. The sandwich configuration of the sample 402, the substrates (e.g., slide 403 and 404) may reduce a burden of users to develop in house tissue sectioning and/or tissue mounting expertise. Further, the sandwich configuration may enable selection of a particular region of interest of analysis (e.g., for a tissue section larger than the substrate comprising a plurality of capture agents). The sandwich configuration also beneficially enables analysis without having to place a biological sample (e.g., tissue section) 402 directly on the substrate comprising a plurality of capture agents (e.g., slide 404).

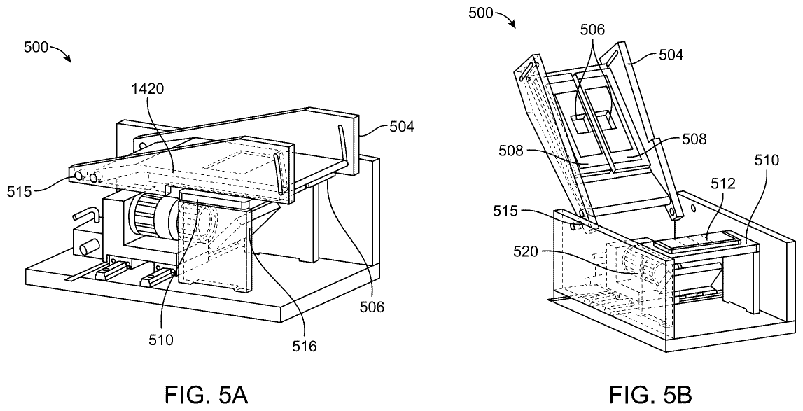

FIG. 5A is a perspective view of an example sample handling apparatus 500 in a closed position in accordance with some example implementations. As shown, the sample handling apparatus 500 includes a first member 504, a second member 510, optionally an image capture device 520, a first substrate 506, optionally a hinge 515, and optionally a mirror 516. The hinge 515 may be configured to allow the first member 504 to be positioned in an open or closed configuration by opening and/or closing the first member 504 in a clamshell manner along the hinge 515. FIG. 5B is a perspective view of the example sample handling apparatus 500 in an open position in accordance with some example implementations. As shown, the sample handling apparatus 500 includes one or more first retaining mechanisms 508 configured to retain one or more first substrates 506. In the example of FIG. 5B, the first member 504 is configured to retain two first substrates 506, however the first member 504 may be configured to retain more or fewer first substrates 506.

In some aspects, when the sample handling apparatus 500 is in an open position, the first substrate 506 and/or the second substrate 512 may be loaded and positioned within the sample handling apparatus 500 such as within the first member 504 and the second member 510, respectively. As noted, the hinge 515 may allow the first member 504 to close over the second member 510 and form a sandwich configuration.

In some aspects, after the first member 504 closes over the second member 510, an adjustment mechanism (not shown) of the sample handling apparatus 500 may actuate the first member 504 and/or the second member 510 to form the sandwich configuration for the permeabilization step (e.g., bringing the substrate 506 and the substrate 512 closer to each other and within a threshold distance for the sandwich configuration). The adjustment mechanism may be configured to control a speed, an angle, a force, or the like of the sandwich configuration.

In some embodiments, the biological sample may be aligned within the first member 504 (e.g., via the first retaining mechanism 508) prior to closing the first member 504 such that a desired region of interest of the sample is aligned to be captured by the plurality of capture agents, e.g., when the substrates are aligned in the sandwich configuration. Such alignment may be accomplished manually (e.g., by a user) or automatically (e.g., via an automated alignment mechanism). After or before alignment, spacers may be applied to the first substrate 506 and/or the second substrate 512 to maintain a minimum spacing between the first substrate 506 and the second substrate 512 during sandwiching. In some aspects, the permeabilization may be applied to the first substrate 506 and/or the second substrate 512. The first member 504 may then close over the second member 510 and form the sandwich configuration.

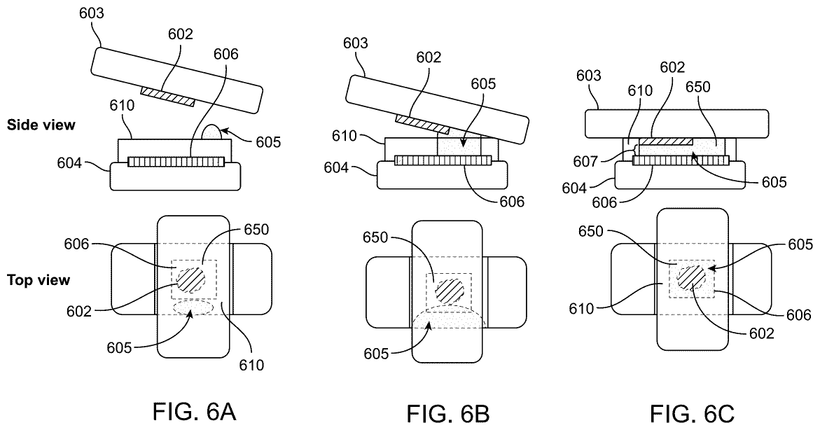

In the example sandwich maker workflows described herein and shown in FIGS. 6A-6C, the reagent medium (e.g., liquid reagent medium, permeabilization solution 605) may fill a gap (e.g., the gap 607) between two substrates (e.g., slide 603 and 604 with capture agents 606) to warrant or enable transfer of molecules. Robust fluidics in the sandwich making described herein may reduce or prevent deflection of molecules as they move from the tissue slide to the capture slide.

FIGS. 6A-6C show an example sandwiching process where a substrate (e.g., slide 603) including a biological sample 602 (e.g., a tissue section), and a substrate (e.g., slide 604 including plurality of capture agents 606) are brought into proximity with one another. A liquid reagent drop (e.g., permeabilization solution 605) is introduced on the substrate in proximity to the capture agents 606 and in between the biological sample 602 and the substrate with the plurality of capture agents. In some embodiments, the permeabilization solution 605 releases analytes that are captured by the capture agents 606. As further shown, one or more spacers 610 may be positioned between the substrates slides 603 and 604. The one or more spacers 610 may be configured to maintain a separation distance between the two substrates. While the one or more spacers 610 is shown as disposed on the substrate 604, the spacer may additionally or alternatively be disposed on the substrate 603.

In some embodiments, the separation distance between first and second substrates is maintained between 2 microns and 1 mm (e.g., between 2 microns and 800 microns, between 2 microns and 700 microns, between 2 microns and 600 microns, between 2 microns and 500 microns, between 2 microns and 400 microns, between 2 microns and 300 microns, between 2 microns and 200 microns, between 2 microns and 100 microns, between 2 microns and 25 microns, between 2 microns and 10 microns), measured in a direction orthogonal to the surface of first substrate that supports sample. In some embodiments, the separation distance between first and second substrates is less than 50 microns. In some instances, the distance is 2 microns. In some instances, the distance is 2.5 microns. In some instances, the distance is about 2, 3, 4, 5, 6, 7, 8, 9, 10, 11, 12, 13, 14, 15, 16, 17, 18, 19, 20, 21, 22, 23, 24, or 25 microns. In some embodiments, the first substrate is placed in direct contact with the sample on the second substrate. In some embodiments, the separation distance is measured in a direction orthogonal to a surface of the second substrate that supports the biological sample.

In some embodiments, the one or more spacers is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of second substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

In some embodiments, the first and second substrates are placed in a substrate holder (e.g., a biological sample/substrate capture area alignment device). In some embodiments, the device comprises a sample holder. In some embodiments, the sample holder includes a first member and a second member that receive a first substrate and a second substrate, respectively. The device can include an alignment mechanism that is connected to at least one of the members and aligns the first and second members. Thus, the devices of the disclosure can advantageously align the first substrate and the second substrate and any samples, immobilized oligonucleotide molecules, or permeabilization reagents that may be on the surface of the first and second substrates.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample is aligned (e.g., vertically aligned) with a portion of the oligonucleotide molecules on the second substrate and within a threshold distance of the lawn of oligonucleotide molecules, and such that the portion of the biological sample and the capture agents contact the reagent medium, wherein the permeabilization reagent releases the target nucleic acids from the biological sample.

In some embodiments of a sample holder, the sample holder includes a first member including a first retaining mechanism configured to retain a substrate comprising a sample. In some embodiments, the first retaining mechanism is configured to retain the substrate disposed in a first plane. The sample holder can further include a second member including a second retaining mechanism configured to retain a second substrate disposed in a second plane. The sample holder further includes an alignment mechanism connected to one or both of the first member and the second member. In some embodiments, the alignment mechanism is configured to align the first and second members along the first plane and/or the second plane such that the sample contacts at least a portion of the reagent medium when the first and second members are aligned and within a threshold distance along an axis orthogonal to the second plane. The alignment mechanism may be configured to move the second member along the axis orthogonal to the second plane and/or move the first member along an axis orthogonal to the first plane.

In some embodiments, the alignment mechanism includes a linear actuator. In some embodiments, the alignment mechanism includes one or more of a moving plate, a bushing, a shoulder screw, a motor bracket, and a linear actuator. The moving plate may be coupled to the first member or the second member. The alignment mechanism may, in some cases, include a first moving plate coupled to the first member and a second moving plate coupled to the second member. In some embodiments, the linear actuator is configured to move the second member along an axis orthogonal to the plane or the first member and/or the second member. For example, the moving plate may be coupled to the second member and adjust the separation distance along a z axis (e.g., orthogonal to the second substrate) by moving the moving plate up in a superior direction toward the first substrate. In some embodiments, the linear actuator is configured to move the first member along an axis orthogonal to the plane of the first member and/or the second member. The movement of the moving plate may be accomplished by the linear actuator configured to move the first member and/or the second member at a velocity. The velocity may be controlled by a controller communicatively coupled to the linear actuator. In some embodiments, the linear actuator is configured to move the first member, the second member, or both the first member and the second member at a velocity of at least 0.1 mm/sec (e.g., at least 0.1 mm/sec to 2 mm/sec). In some aspects, the velocity may be selected to reduce or minimize bubble generation or trapping within the reagent medium. In some embodiments, the linear actuator is configured to move the first member, the second member, or both the first member and the second member with an amount of force of at least 0.1 lbs. (e.g., between 0.1-4.0 pounds of force).

In some aspects, the velocity of the moving plate (e.g., closing the sandwich) may affect bubble generation or trapping within the reagent medium. It may be advantageous to minimize bubble generation or trapping within the reagent medium during the “sandwiching” process, as bubbles can interfere with the migration of the target nucleic acids through the reagent medium to the oligonucleotide lawn. In some embodiments, the closing speed is selected to minimize bubble generation or trapping within the reagent medium. In some embodiments, the closing speed is selected to reduce the time it takes the flow front of the reagent medium from an initial point of contact with the first and second substrate to sweep across the sandwich area (also referred to herein as “closing time”). In some embodiments, the closing speed is selected to reduce the closing time to less than about 1100 ms. In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less.

FIG. 6B shows a fully formed sandwich configuration creating a chamber 650 formed from the one or more spacers 610, the two substrates 603 and 604 in accordance with some example implementations. The liquid reagent (e.g., the permeabilization solution 605) fills the volume of the chamber 650 and the permeabilization buffer allows target nucleic acids to diffuse from the biological sample 602 toward the capture agents 606. In some aspects, flow of the permeabilization buffer may deflect transcripts and/or molecules from the biological sample 602 and may affect diffusive transfer of analytes for analysis. A partially or fully sealed chamber 650 resulting from the one or more spacers 610, the two substrates may reduce or prevent flow from undesirable convective movement of transcripts and/or molecules over the diffusive transfer from the biological sample 602 to the capture agents 606.

FIG. 6A depicts the first substrate (e.g., the slide 603 including biological sample 602) angled over (superior to) the substrate (e.g., slide 604) comprising the plurality of capture agents. As shown, a drop of the reagent medium (e.g., permeabilization solution) 605 is located on the spacer 610 toward the right-hand side of the side view in FIG. 6A. While FIG. 6A depicts the reagent medium on the right hand side of side view, it should be understood that such depiction is not meant to be limiting as to the location of the reagent medium on the spacer.

FIG. 6B shows that as the substrate 603 lowers, and/or as the substrate 604 rises, the dropped side of the substrate 603 (e.g., a side of the slide 603 angled toward the second substrate) may contact the drop of the reagent medium 605. The dropped side may urge the reagent medium 605 toward the opposite direction (e.g., towards an opposite side of the spacer 610, towards an opposite side of the substrate relative to the dropped side, for example from right to left as the sandwich is formed as shown).

In some embodiments, the two substrates are further moved to achieve an approximately parallel arrangement of the first substrate and the second substrate.

FIG. 6C depicts a full closure of the sandwich between the two substrates with the spacer 610 contacting both substrates and maintaining a separation distance and optionally the approximately parallel arrangement between the two substrates.

Further details on support devices, sample holders, sample handling apparatuses, or systems for implementing a sandwiching process are described in, e.g., US 2022/0241780 and WO 2022/061152, each of which are incorporated by reference in their entirety.

In some aspects, it may be possible to reduce or eliminate bubble formation between the slides using a variety of filling methods and/or closing methods. In some embodiments, the substrates are closed at an angle. Further details on angled closure workflows, and devices and systems for implementing an angled closure workflow, are described in WO 2021/252747 and WO 2022/061152, which are hereby incorporated by reference in their entirety.

Additional configurations for reducing or eliminating bubble formation, and/or for reducing unwanted fluid flow, are described in U.S. Patent Application Publication No. 2023/0017773, which is hereby incorporated by reference in its entirety.

Workflows described herein may include contacting a drop of the reagent medium (e.g., liquid reagent medium, e.g., a permeabilization solution) disposed on a first substrate or a second substrate with at least a portion of the second substrate or first substrate, respectively. In some embodiments, the contacting comprises bringing the two substrates into proximity such that the sample on the second substrate is aligned with the oligonucleotide molecules on the first substrate.

In some embodiments, the reagent medium comprises a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., Proteinase K). In some embodiments, the detergent is an anionic detergent (e.g., SDS or N-lauroylsarcosine sodium salt solution). Example permeabilization reagents are described in US. Patent Application Pub. No. 20210189475, which is incorporated by reference in its entirety.

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Example lysis reagents are described in US. Patent Application Pub. No. 20210189475, which is incorporated by reference in its entirety.

In some embodiments, the reagent medium comprises a protease. Example proteases include, e.g., pepsin, trypsin, pepsin, elastase, and Proteinase K. Example proteases are described in US. Patent Application Pub. No. 20210189475, which is incorporated by reference in its entirety.

In some embodiments, the reagent medium comprises a detergent. Example detergents include sodium dodecyl sulfate (SDS), sarkosyl, saponin, Triton X-100™, and Tween-20™. Example detergents are described in US. Patent Application Pub. No. 20210189475, which is incorporated by reference in its entirety.

In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS), Proteinase K, pepsin, N-lauroylsarcosine, RNase, and a sodium salt thereof.

In some embodiments, the reagent medium comprises a permeabilization agent. In some embodiments, the permeabilization agent is a protease (e.g., Proteinase K, trypsin, pepsin, elastase).

The sample holder is compatible with a variety of different schemes for contacting the aligned portions of the biological sample and substrate with the reagent medium to promote capture of the target nucleic acids. In some embodiments, the reagent medium is deposited directly on the second substrate, and/or directly on the first substrate. In some embodiments, the reagent medium is deposited on the first and/or second substrate, and then the first and second substrates aligned in the sandwich configuration such that the reagent medium contacts the aligned portions of the biological sample and substrate. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the second substrate. For example, in some embodiments, a reagent is deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent is applied in dried form directly onto the first substrate or the second substrate or both. In some embodiments, the coating process is done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some embodiments, the workflow includes provision of the second substrate comprising the biological sample. In some embodiments, the workflow includes mounting the biological sample onto the second substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In certain embodiments a dried permeabilization reagent is applied or formed as a layer on the first substrate or the second substrate or both prior to contacting the first and the second substrate. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some instances, the aligned portions of the biological sample and the second substrate are in contact with the reagent medium for about 1 minute. In some instances, the aligned portions of the biological sample and the second substrate are in contact with the reagent medium for about 5 minutes. In some instances, the aligned portions of the biological sample and the second substrate are in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the second substrate are in contact with the reagent medium for about 1-60 minutes. In some instances, the aligned portions of the biological sample and the second substrate are in contact with the reagent medium for about 30 minutes.

In some embodiments, following initial contact between sample and a permeabilization agent, the permeabilization agent can be removed from contact with sample (e.g., by opening sample holder).

In some embodiments, following initial contact between sample and a permeabilization agent, the permeabilization agent can be removed from contact with sample (e.g., by opening sample holder) before complete permeabilization of sample. For example, in some embodiments, only a portion of sample is permeabilized, and only a portion of the analytes in sample may be captured by the immobilized oligonucleotides on the second substrate. In some instances, the reduced amount of the target nucleic acids captured and available for detection can be offset by the reduction in lateral diffusion that results from incomplete permeabilization of sample. In general, the spatial resolution of the assay is determined by the extent of analyte diffusion in the transverse direction (e.g., orthogonal to the normal direction to the surface of sample). The larger the distance between the sample on the second substrate and the immobilized oligonucleotide lawn on the first substrate, the greater the extent of diffusion in the transverse direction, and the concomitant loss of resolution. Analytes liberated from a portion of the sample closest to the immobilized oligonucleotide lawn have a shorter diffusion path, and therefore do not diffuse as far laterally as analytes from portions of the sample farthest from the immobilized oligonucleotide lawn. As a result, in some instances, incomplete permeabilization of the sample (by reducing the contact interval between the permeabilization agent and the sample) can be used to maintain adequate spatial resolution in the assay.

In some instances, after the sandwiching process the two substrates are separated (e.g., such that they are no longer aligned in a sandwich configuration, also referred to herein as opening the sandwich). In some embodiments, subsequent processing and/or analysis can be performed on the captured target nucleic acids after the substrates are separated.

B. Generation of Complements of Captured Target Nucleic Acid on Substrates Comprising Stabilization and/or Interspersing Agents

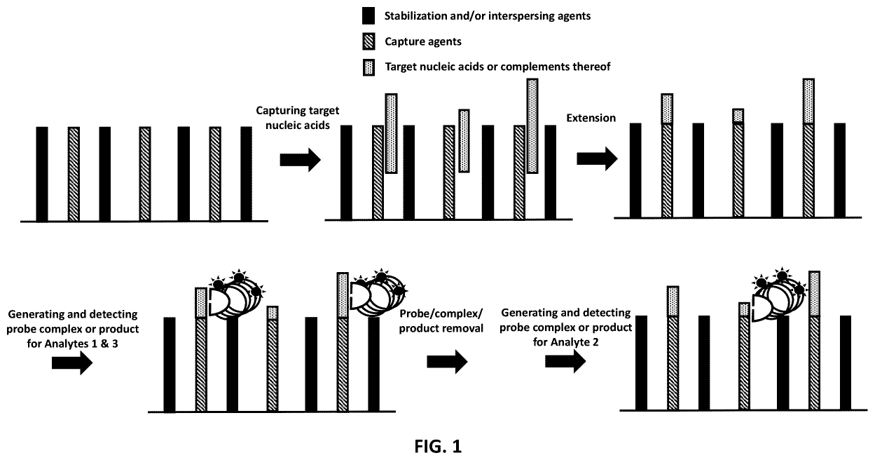

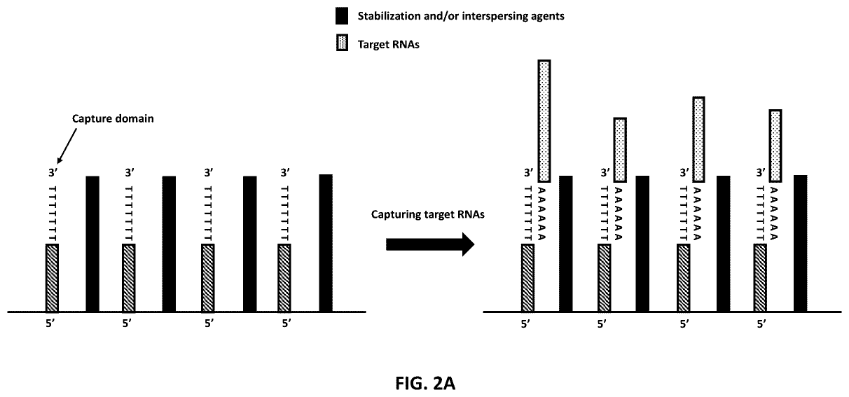

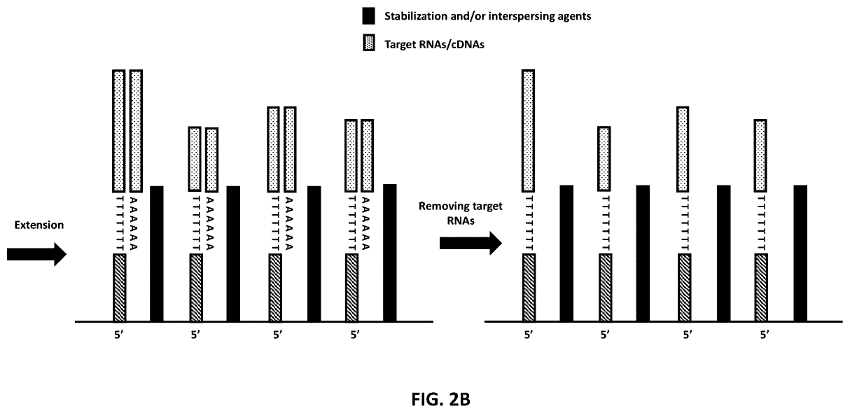

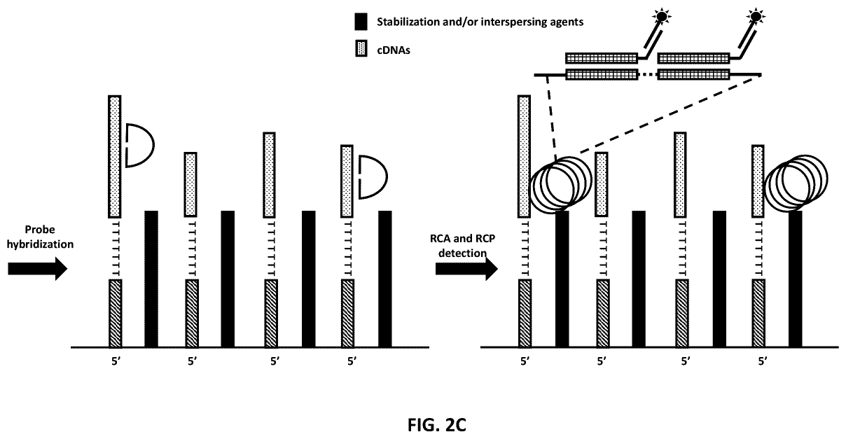

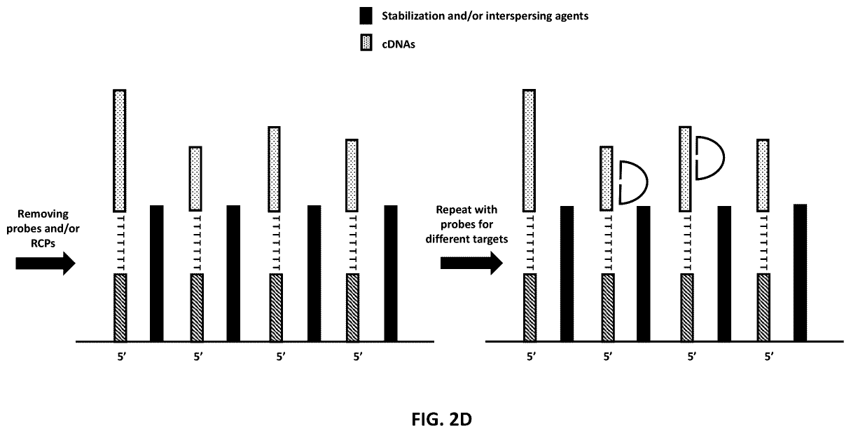

In some embodiments, after a target nucleic acid is captured by a capture agent on a substrate, the capture domain (or a portion thereof) that hybridizes to the captured target nucleic acid is used as a primer that is extended by a polymerase using the captured target nucleic acid as template. As such, a complement of the captured target nucleic acid is generated, transferring sequence information of the target nucleic acid to the immobilized capture agent. The complement immobilized on the substrate (via the capture agent or a portion thereof) is analyzed using a probe or probe set that binds to a sequence in the complement of the captured target nucleic acid, for instance, as shown in FIG. 1 and FIGS. 2A-2D. While poly T (e.g., to capture poly A of mRNA molecules) on the slide is shown in FIGS. 2A-2D, the capture domain can comprise other sequences in the case of capturing the overhang regions of ligated probes generated from templated ligation on a target nucleic acid such as RNA.

In some embodiments, RNA molecules (e.g., mRNA) from a sample are captured by oligonucleotides (e.g., probes comprising a poly(dT) sequence) on a substrate prepared by a method disclosed herein, cDNA molecules are generated via reverse transcription of the captured RNA molecules, and the cDNA molecules (e.g., a first strand cDNA) or portions or products (e.g., a second strand cDNA synthesized using a template switching oligonucleotide) thereof can be detected on the substrate (e.g., using a probe or probe set) and/or separated from the substrate and analyzed (e.g., sequenced).

In some embodiments, the captured RNA is removed from the substrate once the cDNA is generated. In some embodiments, the method comprises removing the captured target nucleic acid or a portion thereof from the substrate, e.g., after generating the complement of the captured target nucleic acid. In some embodiments, the removing comprises digesting the captured target nucleic acid using an enzyme. In some embodiments, the enzyme is an endonuclease, such as RNase H. In some cases, the endonuclease digests RNA sequences that are hybridized in DNA/RNA duplexes, thereby releasing the complement (cDNA) which remains immobilized to the substrate.

In some embodiments, the removing comprises denaturing a duplex formed by the captured target nucleic acid and the complement, and the complement remains immobilized on the substrate via the capture agent after the denaturation. In some embodiments, the method comprises generating a complement of the complement of the captured target nucleic acid. In some embodiments, the method comprises removing the complement of the complement from the substrate for sequence analysis.

In some embodiments, complements of captured target nucleic acids are generated on the substrate comprising stabilization agents and/or interspersing agents. In some embodiments, the stabilization agents and/or interspersing agents do not capture target nucleic acids. In some embodiments, the stabilization agents and/or interspersing agents are blocked from exonuclease cleavage and/or primer extension by a polymerase.

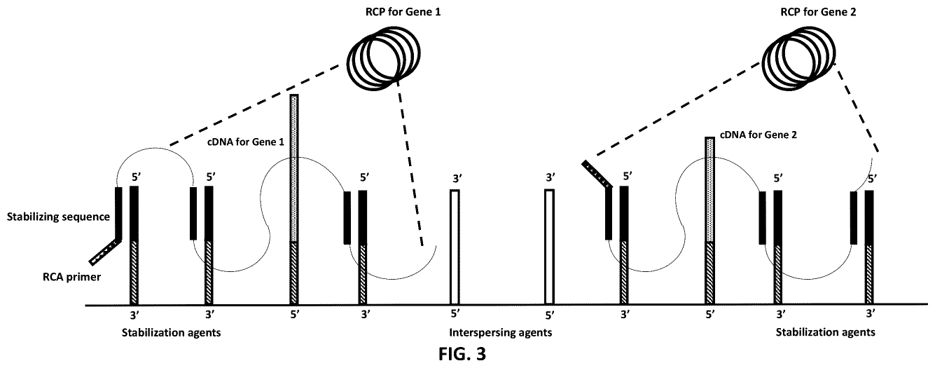

In some embodiments, the capture agents are 5′ immobilized on the substrate. Any one or more of the stabilization agents can be 3′ or 5′ immobilized on the substrate. Any one or more of the interspersing agents can be 3′ or 5′ immobilized on the substrate. In some embodiments, the capture agents are 5′ immobilized on the substrate, and the stabilization and/or interspersing agents are also 5′ immobilized on the substrate. In some embodiments, the capture agents are 5′ immobilized on the substrate, and the stabilization and/or interspersing agents are 3′ immobilized on the substrate. In some embodiments, the capture agents are 5′ immobilized on the substrate, the stabilization agents are 3′ immobilized on the substrate, and the interspersing agents are 5′ immobilized on the substrate, e.g., as shown in FIG. 3.

In some embodiments, any two or more of the capture agents and the stabilization and/or interspersing agents are same or different in length. In some embodiments, the stabilization and/or interspersing agents are longer than the capture agents. Any one or more of the capture agents and the stabilization and/or interspersing agents can be about 10, about 20, about 50, about 100, about 200, about 500, or more nucleotides in length.

In some embodiments, the stabilization agents and/or interspersing agents may “dilute” the capture agents on the substrate, such that the density of the complements (e.g., cDNA) of captured target nucleic acids on the substrate and the distance between adjacent complements on the substrate can be controlled. Optical crowding can be ameliorated by the presence of the stabilization agents and/or interspersing agents, since they do not lead to the generation of detectable nucleic acid sequences that are targeted by the probes or probe sets disclosed herein. In some embodiments, the chances of multiple target nucleic acids being captured by capture agents in close vicinity to each other on the substrate can be reduced by using the stabilization agents and/or interspersing agents.

### C. Probe Hybridization and Ligation

In some aspects, a probe or probe set disclosed herein can be a circularizable probe or probe set. A circularizable probe or probe set can be any nucleic acid molecule or set of nucleic acid molecules that hybridizes to another one or more other nucleic acids such that the ends of the nucleic acid molecule or nucleic acid molecules are juxtaposed or are in proximity for ligation to form a circularized probe (e.g., by ligation with or without gap filling). For example, a circularizable probe may be a padlock probe with ends that can be ligated upon hybridization to a target nucleic acid sequence (or complement thereof) to form a circularized padlock probe. Specific probe designs can vary depending on the application. For instance, probes or probe sets described herein can comprise a circularizable probe or probe set that does not require gap filling to circularize upon hybridization to a template (e.g., a target nucleic acid and/or a probe such as a splint), a gapped circularizable probe or probe set (e.g., one that requires gap filling to circularize upon hybridization to a DNA template).

In some embodiments, the probe or probe set comprises a 3′ overhang and/or a 5′ overhang upon hybridization to the captured target nucleic acid or complement thereof. In some embodiments, the probe or probe set is a circular probe or circularizable probe or probe set. In some embodiments, the circularizable probe or probe set is ligated using the captured target nucleic acid or complement thereof as a template, with or without gap filling prior to the ligation. In some embodiments, the circularizable probe or probe set is ligated using a splint other than the captured target nucleic acid or complement thereof as a template, with or without gap filling prior to the ligation. In some embodiments, the splint hybridizes to the captured target nucleic acid or complement thereof. In some embodiments, the probe or probe set comprises one or more barcode regions.

In some embodiments, the circularizable probe is provided as a single nucleic acid molecule with possible ligation of 3′ and 5′ ends. In some cases, the circularizable probe or probe set is provided as a circularizable probe set in two or more parts, e.g., two, three, four, five or more nucleic acid molecules (a probe set), which create two or more ligation junctions upon hybridization to a target nucleic acid and/or splint. In some embodiments, the method comprises contacting the biological sample with a splint. In some embodiments, a first ligation junction is formed between a first and second probe of a circularizable probe set upon hybridization to a target nucleic acid, and a second ligation junction is formed between the first and second probe upon hybridization of the first and second probe to a splint. Each part of the probe may provide (e.g., form or comprise) 3′ and/or 5′ ends that can be ligated which may be juxtaposed for ligation and be ligated. The 5′ phosphate and 3′ hydroxyl can be ligated together upon hybridization to a target nucleic acid sequence. In some embodiments, the target nucleic acid sequence is in an endogenous nucleic acid analyte (e.g., an endogenous DNA or an endogenous RNA molecule). In some embodiments, the endogenous RNA is an mRNA. In some embodiments, the target nucleic acid sequence is in a probe bound directly or indirectly to an endogenous nucleic acid molecule (e.g., a DNA probe hybridized to an endogenous nucleic acid analyte). In some cases, the target nucleic acid sequence is in a product of an endogenous analyte (e.g., an amplification product). For instance, in some embodiments, an RNA analyte is reverse transcribed to generate a DNA molecule, and the circularizable probe or probe set then hybridizes to the DNA molecule. In some cases, the target nucleic acid sequence is in or is associated with a labeling agent that binds to a non-nucleic acid analyte. In some cases, the circularizable probe or probe set is provided as one or more DNA molecules. In some embodiments, the circularizable probe or probe set is or comprises a DNA/RNA chimera comprising one or more ribonucleotides. In some embodiments, circularizable probe or probe set comprises one or more ribonucleotides at and/or near a 3′ end of the circularizable probe or probe set that can be ligated. In some embodiments, the circularizable probe or probe set comprises a ribonucleotide at its 3′ end. Various example circularizable probes or probe sets are described in U.S. Patent Application Publication No. 2020/0224244, the entire content of which is herein incorporated by reference in its entirety.

In some embodiments, a probe or probe set disclosed herein (e.g., a circularizable probe or probe set) can comprise a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiments, cleavage of the additional sequence 5′ to the first target-specific binding site is performed by a structure-specific cleavage enzyme, e.g., a Flap endonuclease. Suitable Flap endonucleases are described in Ma et al. 2000. JBC 275, 24693-24700 and in U.S. Patent Application Publication No. 2020/022424, the content of which is herein incorporated by reference in its entirety, and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g., dinucleotides or trinucleotides), or cleaving the entire 5′ single-stranded region, e.g., as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In some embodiments, the circularizable probes or probe sets contain one or more barcodes. In some embodiments, one or more barcodes are indicative of (e.g., correspond to) the identity of an analyte or labeling agent in the biological sample. In some embodiments, one or more barcodes are indicative of a sequence in the analyte nucleic acid, such as a single nucleotide (e.g., SNPs or point mutations), a dinucleotide sequence, a short sequence of about 5 nucleotides in length, or a sequence of any suitable length.

In some embodiments, the circularizable probe or probe set includes one or more barcode sequences. In some embodiments, one or more barcode sequences are in the insertion sequence of the selector probe. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the method further comprises prior to the circularizing step, a step of removing molecules of the circularizable probe or probe set that are not bound to the captured target nucleic acid a complement thereof. In some embodiments, the method comprises removing splint molecules not hybridized to the circularizable probe or probe set. In any of the embodiments herein, the method comprises ligating the ends of a circularizable probe or probe set hybridized to a captured target RNA to form a circularized probe. In some embodiments, the hybridization region of the circularizable probe or probe set that hybridizes to a captured target nucleic acid is a contiguous hybridization region, and the circularizable probe or probe set comprises 3′ and 5′ overhangs that hybridize to a splint. In some embodiments, the hybridization region of the circularizable probe or probe set that hybridizes to a captured target nucleic acid is a split hybridization region, and the circularizable probe or probe set comprises 3′ and 5′ overhangs that hybridize to a splint. In any of the embodiments herein, the method comprises ligating the ends of a circularizable probe or probe set hybridized to a splint to form a circularized probe. In some embodiments, a first ligation is performed using the captured target RNA or a complement thereof, and a second ligation is performed using a splint. In some embodiments, a 3′ end and a 5′ end of the circularizable probe or probe set are ligated using the captured target nucleic acid (e.g., RNA) or a complement thereof (e.g., cDNA) as a template.

In some aspects, the circularizable probes or probe sets comprise a hybridization region, wherein the hybridization region on the probe is capable of hybridizing to a hybridization region on the target nucleic acid. In some aspects, the provided methods involve a step of contacting, or hybridizing the circularizable probes or probe sets to a biological sample containing a target nucleic acid in order to form a hybridization complex. Example biological samples are described in Section IV below. In some embodiments, the biological sample is a tissue sample. In some embodiments, the biological sample is a tissue slice or a tissue section. In some embodiments, the biological sample is a fresh frozen tissue sample. In some embodiments, the biological sample is a paraffin embedded formalin fixed (FFPE) tissue sample. In some embodiments, the biological sample is permeabilized. In some embodiments, the method comprises decrosslinking and/or pre-permeabilizing the biological sample before contacting the biological sample with the circularizable probes or probe sets. In some embodiments, the biological sample is permeabilized with a low concentration of Proteinase K.

In some aspects, the biological sample is on a substrate. In some cases, the tissue sample is on the substrate comprising the immobilized oligonucleotide molecules on a capture area of the substrate. The capture are is an area of the substrate that is aligned with the biological sample, so that target nucleic acids migrated from the biological sample toward the substrate can be captured by immobilized oligonucleotide molecules in the capture area. In some instances, the tissue sample is on a first substrate and a second substrate is provided comprising the immobilized oligonucleotide molecules on a surface of the second substrate. In some embodiments, the first substrate and/or the second substrate comprises a substantially flat planar surface. The first substrate and/or the second substrate can be a slide, e.g., a glass slide. For example, a glass slide such as a cover slip may be used. In some embodiments, the first substrate and/or the second substrate is/are optically transparent.

The circularizable probes or probe sets hybridize to target nucleic acid sequences in the biological sample. In some embodiments, the method comprises incubating the sample under conditions optimized for hybridization of the circularizable probes or probe sets to their corresponding target nucleic acid sequences in the biological sample. In some embodiments, the circularizable probes or probe sets are hybridized to the target sequences at a temperature between about 30° C. and about 60° C. (e.g., between about 30° C. and about 50° C.). In some embodiments, the method can further comprise prior to the ligating step, a step of removing molecules of the circularizable probe or probe set that are not bound to the target nucleic acid from the biological sample.

In some embodiments, the hybridization comprises the pairing of substantially complementary or complementary nucleic acid sequences between circularizable probes or probe sets and target nucleic acids. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another. In some embodiments, the method comprises hybridizing the circularizable probes or probe sets to target nucleic acid sequences at conditions optimized for hybridization of circularizable probes or probe sets to fully complementary target nucleic acid sequences. In some cases, the method comprises performing one or more post-hybridization washes. In some embodiments, the one or more post-hybridization washes are performed under stringent conditions.

In some aspects, the provided methods comprise one or more steps of ligating a circularizable probe or probe set to form a circularized probe hybridized to a capture nucleic acid or a complement thereof immobilized on a substrate. In some embodiments, the ligation involves two or more ligations. In some embodiments, the ligation involves chemical ligation (e.g., click chemistry ligation). In some embodiments, the ligation involves template dependent ligation. In some embodiments, the ligation involves template independent ligation. In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76 (14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a direct ligation. In some embodiments, the ligation herein is an indirect ligation. In a direct ligation, the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps.” In some embodiments, said ends are not ligated directly to each other, but instead are ligated indirectly either via the intermediacy of one or more intervening (so-called “gap” or “gap-filling” (oligo) nucleotides) or by the extension of the 3′ end of a probe to “fill” the “gap’ corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some embodiments, the ligation herein is preceded by gap filling. In other embodiments, the ligation herein does not require gap filling.

In some embodiments, one or more wash steps are performed after ligating the circularizable probes or probe sets to remove unbound probes from the substrate. In some cases, the one or more wash steps is/are performed under stringent conditions. In some embodiments, the one or more wash steps is/are performed using phosphate buffered saline comprising a non-ionic surfactant. In some embodiments, the wash buffer is PBST.

### D. On-Substrate Probe Complex/Product Generation

In some embodiments, a method disclosed herein may also comprise one or more signal amplification components, for instance, by on-substrate generation of probe complexes and/or products such as RCPs. In some embodiments, the present disclosure relates to the detection of nucleic acid sequences in situ using probe hybridization and generation of amplified signals associated with the probes, wherein background signal is reduced and sensitivity is increased. Example signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in U.S. Patent Application Publication No. 2019/0055594 incorporated herein by reference), hybridization chain reaction (HCR), linear oligonucleotide hybridization chain reaction (LO-HCR), assembly of topologically catenated DNA structures using serial rounds of chemical ligation (clampFISH), signal amplification via hairpin-mediated concatemerization (e.g., as described in U.S. Patent Application Publication No. 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used. For instance, HCR is described in detail in Dirks and Pierce, 2004, PNAS, 101 (43), 15275-15278 and in U.S. Pat. No. 7,632,641 (see also U.S. Patent Application Publication No. 2006/00234261; Chemeris et al., 2008 Doklady Biochemistry and Biophysics, 419, 53-55; Niu et al., 2010, 46, 3089-3091; Choi et al., 2010, Nat. Biotechnol. 28 (11), 1208-1212; and Song et al., 2012, Analyst, 137, 1396-1401), which are incorporated herein by reference. Example methods and compositions for LO-HCR are described in U.S. Patent Application Publication No. 2021/0198723, incorporated herein by reference in its entirety. For example branched signal amplification, see e.g., U.S. Patent Application Publication No. US20200399689A1 and Xia et al., Multiplexed Detection of RNA using MERFISH and branched DNA amplification. Scientific Reports (2019), each of which is fully incorporated by reference herein.

In some embodiments, provided herein are methods for performing amplification on the substrate using the captured target nucleic acids or complements thereof. In some embodiments, the amplification product is a rolling circle amplification product (RCP), e.g., an RCP comprising multiple copies of the complement of a barcode region in the probe or probe set. In some embodiments, the 3′ end of the complement of the captured target nucleic acid is blocked from exonuclease digestion and primer extension, and the RCP is generated using a primer distinct from the complement of the captured target nucleic acid. In some embodiments, the 3′ end of the complement of the captured target nucleic acid is unblocked from exonuclease digestion and primer extension, and the RCP is generated using the complement or a portion thereof as a primer.

In some embodiments, the methods disclosed herein include performing rolling circle amplification (RCA) to generate a rolling circle amplification product (RCP) in the absence of a target nucleic acid and a biological sample. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. (See, e.g., Baner et al., Nucl. Acids Res. 26:5073-5078, 1998; Lizardi et al., Nature Genetics 19:226, 1998; Mohsen et al., Acc Chem Res. 2016 Nov. 15; 49 (11): 2540-2550; Schweitzer et al. Proc. Natl Acad. Sci. USA 97:101 13-1 19, 2000; Faruqi et al., BMC Genomics 2:4, 2000; Nallur et al., Nucl. Acids Res. 29: el 18, 2001; Dean et al. Genome Res. 1 1:1095-1099, 2001; Schweitzer et al., Nature Biotech. 20:359-365, 2002; U.S. Pat. Nos. 6,054,274, 6,291,187, 6,323,009, 6,344,329 and 6,368,801). In some embodiments, the capture domain comprised by a capture agent is used as a primer for primer extension using the captured target nucleic acid as a template.

In any of the embodiments herein, the RCA comprises a linear RCA, a branched RCA, a dendritic RCA, or any combination thereof. In any of the preceding embodiments, the amplification product is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiment, the polymerase is Phi29 DNA polymerase.

In some embodiments, upon addition of a DNA polymerase in the presence of appropriate dNTP precursors and other cofactors, the capture domain of an immobilized oligonucleotide primes elongation to produce multiple copies of the circular template. This amplification step can utilize isothermal amplification or non-isothermal amplification.

In some embodiments, RCA is performed in a buffer comprising a crowding agent. In some embodiments, the crowding agent is selected from the group comprising poly(ethylene glycol) (PEG), glycerol, Ficoll, and dextran sulfate. In some embodiments, the crowding agent is poly(ethylene glycol) (PEG). the PEG is selected from the group consisting of PEG200, PEG8000, and PEG35000. In some embodiments, the buffer comprises between about 5% and about 15% PEG. In some embodiments, the buffer comprises about 10% PEG.

In some embodiments, RCA is performed for no more than 60 minutes. In some embodiments, RCA is performed for no more than 90 minutes. In some embodiments, RCA is performed for no more than 120 minutes.

In some embodiments, RCA is performed at a temperature between or between about 20° C. and about 60° C. In some embodiments, RCA is performed at a temperature between or between about 30° C. and about 40° C. In some aspects, RCA is performed at a temperature between at or about 25° C. and at or about 45° C., such as at or about 25° C., 27° C., 29° C., 31° C., 33° C., 35° C., 37° C., 39° C., 41° C., 43° C., or 45° C.

In any of the preceding embodiments, the amplification product can be immobilized on the substrate for subsequent processing, detection and analysis. The methods provided herein can preserve the spatial localization of the rolling circle amplification products on the substrate, which can be correlated with the location in the sample.

In some embodiments, the RCP can be hybridized to one or more of the plurality of stabilization agents via one or more of the stabilizing sequences in the RCP. The lengths of stabilizing sequences is about 10, about 15, about 20, about 25, about 30, about 35, about 40 nucleotides or longer. In some embodiments, the length of stabilizing sequences is between 10-50 nucleotides, between 10-40 nucleotides, between 10-30 nucleotides, between 10-20 nucleotides, between 20-50 nucleotides, between 20-40 nucleotides, between 20-30 nucleotides, or between 30-40 nucleotides. For instance, as shown in FIG. 3, RCPs of different target nucleic acids bind to one or more of the plurality of stabilization agents in the vicinity of the RCP, thereby stabilizing the RCPs and limiting their movement on the substrate during sequential cycles of probe hybridization and detection. In some embodiments, adjacent RCPs are spaced apart from each other by one or more of the plurality of interspersing agents, e.g., to reduce optical crowding, as shown in FIG. 3.

In some embodiments, the method comprises crosslinking the RCP to the stabilization agents and/or to the capture agents after the RCP hybridizing to the one or more stabilization agents. In some embodiments, the method does not comprise crosslinking the RCP to the stabilization agents and/or to the capture agents.

### E. Detection and Analysis

In some aspects, the methods disclosed herein further comprise detecting RCA products (RCPs) immobilized on a substrate. In some cases, the methods also comprise imaging the biological sample (e.g., on a different substrate prior to transfer of the target nucleic acids to the substrate comprising capture agents, or on the same substrate prior to removal of the biological sample). In some embodiments, the methods comprise overlaying or superimposing data obtained from imaging the biological sample with data obtained from detecting the rolling circle amplification products on the substrate. For example, in some cases tissue segmentation information (e.g., based on H&E staining or immunofluorescence) is overlaid with the detected RCPs to correlate spatial localizations of RCPs on the substrate with the position of the corresponding analytes in the biological sample. In some cases, one or more fiducial markers are used to help correlate the positions in the biological sample with positions of RCPs on the substrate. In some embodiments, a plurality of images of the biological sample is acquired. For examples, one or more images of the biological sample stained with a nuclear stain, a histological stain, and/or an immunologic stain can be acquired and one or more images can be acquired to detect RCPs in the biological sample. In some aspects, the plurality of images can be acquired separately.

### (i). Imaging of Biological Samples

In some aspects, the methods provided herein comprise imaging a biological sample. In some instances, an image of a biological sample is analyzed in combination with data from on-slide detection of amplification products. In some embodiments, the biological sample is imaged prior to contacting the sample with circularizable probes or probe sets. In some embodiments, the biological sample is imaged after contacting the sample with circularizable probes or probe sets. In some embodiments, the biological sample is stained prior to imaging. In some embodiments, the biological sample is stained with a nuclear stain, a histological stain, and/or an immunologic stain. In some embodiments, the biological sample is stained with hematoxylin and eosin (H&E) stain.

To facilitate visualization, biological samples can be stained using a wide variety of stains and staining techniques. In some embodiments, for example, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g., DiI, DiO, DiR, DiD). Other cell membrane stains may include FM and RH dyes or immunohistochemical reagents specific for cell membrane proteins. In some examples, the stain may include, including but not limited to, acridine orange, Bismarck brown, carmine, Coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with hematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, the imaging is performed using a microscopy method such as bright field microscopy (e.g., to detect H&E staining), oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the biological sample is imaged by confocal microscopy (for example, to detect immunofluorescent staining such as for identifying cell structures, protein expression, or cell boundaries). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity, thus long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition, and results in a higher quality of generated data. In some embodiments, the biological sample is imaged by fluorescent microscopy and/or brightfield microscopy.

### (ii). Detecting Rolling Circle Amplification Products

In any of the embodiments herein, the detecting comprises detecting RCPs in the absence of a biological sample (e.g., generated using circularized probes captured on the substrate). In any of the embodiments herein, the detecting comprises detecting a signal associated with a fluorescently labeled probe that directly or indirectly binds to a RCP of the circularized probe. In any of the embodiments herein, the sequence of RCP or other generated product is analyzed by sequential hybridization, sequencing by hybridization, sequencing by ligation, sequencing by synthesis, sequencing by binding, or a combination thereof.

In some embodiments, the RCPs are detected as fluorescent spots on the substrate. In some embodiments, the diameters of at least 80%, at least 90%, or at least 95% of the rolling circle amplification products (RCP) (e.g., the diameters of the detected fluorescent spots) are within 3 μm of the median detected RCP diameter.

In some embodiments, the RCP is detected using nucleic acid probes that hybridize to the RCP at sequences other than the stabilizing sequences. In some embodiments, the nucleic acid probes hybridize to barcode sequences in the RCP.

In any of the embodiments herein, the detecting step comprises contacting the substrate with one or more detectably-labeled probes that directly or indirectly bind to the rolling circle amplification product. In some embodiments, the method comprises removing (e.g., dehybridizing) the one or more detectably-labeled probes from the rolling circle amplification product. In any of the embodiments herein, the contacting and removing (e.g., dehybridizing) steps can be repeated with the one or more detectably-labeled probes and/or one or more other detectably-labeled probes that directly or indirectly bind to the rolling circle amplification product.

In some embodiments, the detectably labeled probes bind to one or more intermediate probes comprising one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the substrate with a pool of intermediate probes corresponding to different barcode sequences or portions thereof, and a pool of detectably-labeled probes corresponding to different detectable labels. In some embodiments, the substrate is sequentially contacted with different pools of intermediate probes. In some instances, a common or universal pool of detectably-labeled probes is used in a plurality of sequential hybridization steps (e.g., with different pools of intermediate probes).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, an N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Patent Application Publication No. 20190055594 and U.S. Patent Application Publication No. 20210164039, which are hereby incorporated by reference in their entirety.

In any of the embodiments herein, the detecting step comprises contacting the substrate with one or more intermediate probes that directly or indirectly bind to the rolling circle amplification product, wherein the one or more intermediate probes are detectable using one or more detectably-labeled probes. In some embodiments, the intermediate probe comprises: (i) a recognition sequence complementary to a barcode sequence or portion thereof in the rolling circle amplification product, and (ii) a binding site for the detectably labeled probe. In any of the embodiments herein, the detecting step can further comprise removing (e.g., dehybridizing) the one or more intermediate probes and/or the one or more detectably-labeled probes from the rolling circle amplification product. In any of the embodiments herein, the contacting and removing (e.g., dehybridizing) steps can be repeated with the one or more intermediate probes, the one or more detectably-labeled probes, one or more other intermediate probes, and/or one or more other detectably-labeled probes.

In some embodiments, the target nucleic acid is a first target nucleic acid and the probe or probe set is a first probe or probe set, and the method comprises: contacting the substrate with a second probe or probe set that binds to a second captured target nucleic acid or a complement thereof, wherein the first and second target nucleic acids are different; generating a second amplification product associated with the second probe or probe set, wherein the second amplification product comprises a plurality of stabilizing sequences complementary to a sequence of each of the plurality of stabilization agents; and detecting a signal associated with the second amplification product, thereby detecting the second target nucleic acid in the biological sample.

In some embodiments, the target nucleic acid is a first target nucleic acid and the probe or probe set is a first probe or probe set, and the method comprises: contacting the substrate with a second probe or probe set that binds to a second captured target nucleic acid or a complement thereof, wherein the first and second target nucleic acids are different; and detecting a signal associated with the second probe or probe set or a complex or product thereof, thereby detecting the second target nucleic acid in the biological sample.

For example, as shown in FIG. 1 and FIGS. 2A-2D, the method comprises removing the first probe or probe set, the complex or product of the first probe or probe set, and/or one or more nucleic acid probes that bind to the first probe or probe set or the complex or product thereof for detecting the signal associated therewith, prior to contacting the substrate with the second probe or probe set. In some embodiments, the removing comprises denaturation using heat denaturation, a denaturing agent, enzymatic cleavage, and/or chemical cleavage. In some embodiments, the nucleic acid probes hybridize to barcode sequences in the complex or product (e.g., RCP).

In any of the embodiments herein, the detecting of RCPs is correlated with imaging of the biological sample as described herein, to determine the locations of corresponding analytes in the biological sample. In some embodiments, the data can be overlaid with the help of fiducial marks on the substrate.

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, all or a portion of the RCP (e.g., a barcode sequence in the RCP) is detected by sequencing on the substrate. In some embodiments, sequencing of RCPs on the substrate is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675, 5,750,341, 6,969,488, 6,172,218, and 6,306,597, all of which are herein incorporated by reference in their entireties.

In some embodiments, nucleic acid hybridization is used for sequencing RCPs on the substrate. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, sequence analysis of RCPs on the substrate is performed by sequential hybridization (e.g., sequencing by hybridization and/or sequential in situ fluorescence hybridization). Sequential fluorescence hybridization involves sequential hybridization of detection probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Example methods comprising sequential fluorescence hybridization of detectable probes are described in U.S. Patent Application Publication Nos. 2019/0161796, 2020/0224244, 2022/0010358, 2021/0340618, and 2023/0039899, all of which are incorporated herein by reference. In some aspects, detection that involves repeated cycles of probe hybridization and probe removal may benefit from the methods provide herein which preserve the spatial localization of the rolling circle amplification products on the substrate such that it can be probed repeated with detectably labeled probes.

In some embodiments, sequence analysis is performed by using a base-by-base sequencing method, e.g., sequencing-by-synthesis (SBS), sequencing-by-avidity (SBA), or sequencing-by-binding (SBB). In some embodiments, the sequence to be analyzed is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

In some embodiments, for sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, U.S. Patent Application Publication Nos. 2007/0166705, 2006/0188901, U.S. Pat. No. 7,057,026, 2006/0240439, 2006/0281109, 2011/0059865, 2005/0100900, U.S. Pat. No. 9,217,178, US 2009/0118128, 2012/0270305, 2013/0260372, and 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g., a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e., different types of ternary complexes differing in the type of nucleotide it contains) or by separately delivering the reagents needed to form each type of ternary complex. In some instances, the labeling comprises fluorescence labeling, e.g., of the cognate nucleotide or the polymerase that participate in the ternary complex.

In some embodiments, sequencing is performed by sequencing-by-avidity (SBA). Some aspects of SBA approaches are described in U.S. Patent Application Publication No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, real-time monitoring of DNA polymerase activity is used during sequencing of RCPs on the substrate. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

### IV. Samples, Analytes, and Target Sequences

In some aspects, provided herein are methods for detecting one or more target nucleic acid sequences in a biological sample.

### A. Samples

A sample disclosed herein can be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO), or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). In some embodiments, the biological sample includes nucleic acids (such as DNA or RNA). In some embodiments, the biological sample is obtained from a tissue sample, such as a tissue section, a biopsy, a core biopsy, a needle aspirate, or a fine needle aspirate. In some embodiments, the sample is a skin sample, a colon sample, a histology sample, a histopathology sample, a tumor sample, living cells, cultured cells, or a clinical sample such as, for example, cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms, for example, in a community or ecosystem.

In some embodiments, biological samples include one or more diseased cells. A diseased cell can have altered metabolic properties, gene expression, protein expression, and/or morphologic features. Examples of diseases include inflammatory disorders, metabolic disorders, nervous system disorders, and cancer. In some embodiments, cancer cells are derived from solid tumors, hematological malignancies, cell lines, or obtained as circulating tumor cells. In some embodiments, biological samples also include fetal cells and immune cells.

In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

A variety of steps can be performed to prepare or process a biological sample for and/or during an assay. Except where indicated otherwise, the preparative or processing steps described below can generally be combined in any manner and in any order to appropriately prepare or process a particular sample for analysis.

### (i) Sample Permeabilization and Treatment

In some embodiments, a biological sample can be permeabilized to facilitate transfer of analytes or products thereof (such as the target nucleic acids) out of the sample, and/or to facilitate transfer of species (such as probes) into the sample.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by adding one or more lysis reagents to the sample. Examples of suitable lysis agents include, but are not limited to, bioactive reagents such as lysis enzymes that are used for lysis of different cell types (e.g., gram positive or negative bacteria, plants, yeast, mammalian), such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample is permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as Proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, Proteinase K treatment may be used to free up DNA with proteins bound thereto.

### (ii) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

(iii) Freezing, Fixation, and Postfixation

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than-20° C., or less than −25° C.

In some embodiments, the biological sample is prepared using formalin-fixation and paraffin-embedding (FFPE), which are established methods. In some embodiments, cell suspensions and other non-tissue samples is prepared using formalin-fixation and paraffin-embedding. Following fixation of the sample and embedding in a paraffin or resin block, the sample is sectioned as described above. Prior to analysis, the paraffin-embedding material is removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more circularizable probes or probe sets. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., 347 (6221): 543-548, 2015, the entire contents of which are incorporated herein by reference.

### (v) Crosslinking and De-Crosslinking

In some embodiments, the biological sample is reversibly cross-linked prior to or during an in situ assay module (e.g., imaging the biological sample prior to transfer of the target nucleic acids to the substrate). In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof are modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the modified probe to the matrix.

In some embodiments, the biological sample is immobilized in a hydrogel via cross-linking of the polymer material that forms the hydrogel. In some embodiments, cross-linking is performed chemically and/or photochemically, or alternatively by any other suitable hydrogel-formation method. A hydrogel may include a macromolecular polymer gel including a network. In some embodiments, within the network, some polymer chains can be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes and dNTPs used to amplify the nucleic acid and to attach the barcode to the amplified fragments. Other enzymes can be used, including without limitation, RNA polymerase, ligase, Proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### (vi) Tissue Permeabilization and Treatment

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by adding one or more lysis reagents to the sample. In some embodiments, processing of the biological sample, such as de-waxing, allows the biological sample to become permeabilized. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample is permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as Proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, Proteinase K treatment may be used to free up DNA with proteins bound thereto.

(vii) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

### B. Analytes

In any of the embodiments herein, the methods and compositions disclosed herein are used to detect and analyze a wide variety of different analytes e.g., using the probes described in Section III. In some aspects, an analyte includes any biological substance, structure, moiety, or component to be analyzed. In some aspects, a target disclosed herein may similarly include any analyte of interest. In some examples, a target or analyte can be directly or indirectly detected.

In some of the embodiments, analytes are derived from a specific type of cell and/or a specific sub-cellular region. In some embodiments, analytes are derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

An analyte can be any substance or entity for which a specific binding partner (e.g., an affinity binding partner) can be developed. Such a specific binding partner may be a nucleic acid probe (for a nucleic acid analyte) and may lead directly to the generation of a RCA template (e.g., a padlock or other circularizable probe or probe set). Alternatively, the specific binding partner may be coupled to a nucleic acid, which may be detected using an RCA strategy, e.g., in an assay which uses or generates a circular nucleic acid molecule which can be the RCA template.

Analytes of particular interest may include nucleic acid molecules, such as DNA (e.g., genomic DNA, mitochondrial DNA, plastid DNA, viral DNA, etc.) and RNA (e.g., mRNA, microRNA, rRNA, snRNA, viral RNA, etc.), and synthetic and/or modified nucleic acid molecules, (e.g., including nucleic acid domains comprising or consisting of synthetic or modified nucleotides such as LNA, PNA, morpholino, etc.).

### (i) Endogenous Analytes

In some embodiments, an analyte herein is endogenous to a biological sample and can include nucleic acid analytes and non-nucleic acid analytes. Methods and compositions disclosed herein are used to analyze nucleic acid analytes (e.g., using a circularizable nucleic acid probe or probe set as described in Section III that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Example analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, SiRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte is a denatured nucleic acid, wherein the resulting denatured nucleic acid is single-stranded. In some embodiments, the nucleic acid is denatured using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent includes an agent that interacts with an analyte (e.g., an endogenous analyte in a sample). In some embodiments, the labeling agents comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds is also identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of example labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. In some embodiments, the antibodies or epitope-binding fragments including the analyte binding moiety specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the optical detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent comprises a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample are subsequently associated with the one or more physical properties of the biological sample. For example, the multiple different species of analytes can be associated with locations of the analytes in the biological sample. Such information (e.g., proteomic information when the analyte binding moiety (ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### C. Target Sequences

A target sequence for a probe disclosed herein (e.g., a circularizable probe or probe set as described in Section III) may be comprised in or associated with any analyte disclose herein, including an endogenous analyte (e.g., a cellular nucleic acid).

In some embodiments described herein, the analyte comprises or is associated with a target sequence. In some embodiments, a target sequence for a nucleic acid probe described herein is a marker sequence for a given analyte. A marker sequence is a sequence that identifies a given analyte (e.g., alone or in combination with one or more other marker sequences). Thus, in some embodiments, a marker sequence for a given target analyte is specific to that analyte, or unique, such that multiple target analytes can be distinguished from each other.

A “marker sequence” is thus a sequence which marks, is associated with, or identifies a given analyte. It is a sequence by which a given analyte may be detected and distinguished from other analytes. Where an “analyte” comprises a group of related molecules e.g., isoforms or variants or mutants etc., or molecules in a particular class or group, it is not required that a marker is unique or specific to only one particular analyte molecule, and it may be used to denote or identify the analyte as a group. However, where desired, a marker sequence may be unique or specific to a particular specific analyte molecule, e.g., a particular variant. In this way different variants, or isoforms, or mutants may be identified or distinguished from one another.

Where the analyte is a nucleic acid molecule, the target sequence (e.g., a marker sequence) may be a sequence present in the target analyte molecule, or a complement thereof (e.g., a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte as a tag or identifier (ID) sequence (e.g., a barcode) for the analyte or labeling agent. It may thus be a synthetic or artificial sequence.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) also provides a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

### V. Compositions and Kits

Also provided herein are kits, for example comprising one or more polynucleotides disclosed herein, and reagents for performing the methods provided herein, for example reagents required for one or more steps comprising hybridization, ligation, amplification, detection, sequencing, immobilized oligonucleotide coated-substrate preparation, and/or sample preparation as described herein. In some embodiments, the kit comprises one or more substrates (e.g., a first substrate and/or a second substrate). For example, the substrate comprises a plurality of capture agents, a plurality of interspersing agents, and a plurality of stabilization agents immobilized thereon, wherein each capture agent comprises a capture domain capable of capturing a target nucleic acid of a plurality of target nucleic acids.

In some examples, a substrate may comprise a plurality of capture agents (e.g., capture probes) directly or indirectly immobilized thereon. In some embodiments, the kit further comprises a target nucleic acid. In some embodiments, any or all of the polynucleotides are DNA molecules. In some embodiments, the target nucleic acid is a messenger RNA molecule. In some embodiments, the kit comprises one or more reagents for migration of the target nucleic acids (e.g., the reagent medium) as described in Section III. In some embodiments, the kit comprises one or more reagents for staining the biological sample. In some aspects, the kit comprises a plurality of probes or probe sets, wherein each probe or probe set binds to a target nucleic acid of the plurality of target nucleic acids or a complement of the target nucleic acid, and the probes or probe sets for different target nucleic acids share a common sequence.

The various components of the kit may be present in separate containers or certain compatible components may be pre-combined into a single container. In some embodiments, the kits further contain instructions for using the components of the kit to practice the provided methods.

In some embodiments, the kits contains reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit also comprises any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

In some embodiments, provided herein are systems for performing the methods described herein including support devices, sample holders, sample handling apparatuses, or systems for implementing a sandwiching process are described in, e.g., US. Patent Application Pub. No. 20210189475, and WO 2022/061152, each of which are incorporated by reference in their entirety.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.”

A “hybridization complex” as used herein may comprise one, two, or more strands or separate molecules. A hybridization complex that comprises three or more strands or separate molecules does not necessarily comprise direct hybridization between every possible pairwise combination thereof, so long as at least two molecules or strands are directly hybridized to each other, or are in the process of binding to or unbinding from each other, at a given time.

In general, the stability of a hybrid is a function of the ion concentration and temperature. Typically, a hybridization reaction is performed under conditions of lower stringency, followed by washes of varying, but higher, stringency. Example stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanchisa, Nucleic Acids Res. 12:203 (1984).

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” refers, in some embodiments, to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present disclosure and their respective alleles may be derived from any number of sources, such as public databases (U.C. Santa Cruz Human Genome Browser Gateway (genome.ucsc.edu/cgi-bin/hgGateway) or the NCBI dbSNP website (www.ncbi.nlm.nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations. It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “adjacent” as used herein includes but is not limited to being directly linked by a phosphodiester bond. For example, “adjacent” nucleotides or regions on a nucleic acid such as a probe may be separated by a number of nucleotides. For instance, a toehold region and an interrogatory region adjacent to each other may be separated by 0, 1, 2, 3, 4, 5, 6, 7, 8, 9, 10 or more nucleotides in a probe.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

The term “each,” when used in reference to a collection of items, is intended to identify an individual item in the collection but does not necessarily refer to every item in the collection, unless expressly stated otherwise, or unless the context of the usage clearly indicates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Analyte Detection Using a Substrate of Immobilized Capture Oligonucleotide Agents and Stabilization Agents

This example describes a general workflow for analyzing a biological tissue sample on a substrate in the presence of stabilization agents and/or interspersing agents in a lawn of immobilized capture agents on the substrate, which can provide amplification products with high positional stability (e.g., during probe hybridization and signal detection cycles) and mitigate autofluorescence observed in biological samples.

A substrate comprising a plurality of immobilized oligonucleotide molecules, including a plurality of capture agents each comprising a capture domain, a plurality of stabilization and/or interspersing agents is prepared. For example, the ratio of capture agents to stabilization agents on the substrate is 100:1 and interspersing agents are included on the substrate such that capture agents are spaced apart from one another to reduce the density of RCPs on the substrate when generated. Paraffin embedded formalin fixed (FFPE) mouse brain tissue samples are sectioned, the tissue is permeabilized and target nucleic acids (e.g., RNA) or hybridized ligated linear probes are migrated toward the substrate with a plurality of capture agents by diffusion.

After capture of the target nucleic acids, the substrate is then contacted with a circularizable probe or probe set that binds to the captured target nucleic acid or a complement thereof. Circularizable probes for a panel of target genes are added to the sample and allowed to hybridize. Samples are washed to remove unbound probe. For probe ligation, a ligation reaction mix including T4 ligase buffer and T4 RNA ligase is added to the sample. For RCA, samples are incubated in a reaction mix containing Phi29 polymerase buffer, dNTPs, and Phi29 polymerase. RCA products (RCP) also include a plurality of copies of the stabilizing sequences complementary to a sequence of each of the plurality of stabilization agents on the substrate. The immobilized and stabilized RCPs are detected by hybridizing labeled probes to the RCPs in situ and imaging samples with a fluorescent microscope. In some cases, the circularized probes and generated products can be stripped and removed from the slide and the protocol can be repeated with a different panel of genes for detection.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-305. (canceled)

306. A method for analyzing a biological sample, comprising:
a) contacting the biological sample comprising cells with a substrate, wherein:
the biological sample comprises a plurality of target nucleic acids,
the substrate comprises a plurality of capture agents and a plurality of stabilization agents, and
each capture agent is a nucleic acid comprising a capture domain capable of capturing a target nucleic acid of the plurality of target nucleic acids;
b) providing conditions to allow the capture agents to capture the target nucleic acids;
c) contacting the substrate with a probe or probe set that binds to the captured target nucleic acid or a complement thereof;
d) generating an amplification product associated with the probe or probe set, wherein the amplification product comprises a plurality of stabilizing sequences complementary to a sequence of each of the plurality of stabilization agents; and
e) detecting a signal associated with the amplification product on the substrate, wherein the location of the signal on the substrate corresponds to the location of the target nucleic acid in the biological sample.

307. The method of claim 306, wherein the biological sample is immobilized on the substrate, or wherein the substrate is a first substrate and the biological sample is immobilized on a second substrate and the first and second substrates are aligned with each other during the contacting in a).

308. The method of claim 306, wherein the plurality of capture agents and/or the plurality of stabilization agents are randomly distributed on the substrate or in a capture area on the substrate.

309. The method of claim 306, wherein the density of molecules of the plurality of capture agents and molecules of the plurality of stabilization agents on the substrate or in a capture area on the substrate is between 1 and 10 picomoles per 10 μm² area.

310. The method of claim 306, wherein molecules of the plurality of capture agents and/or molecules of the plurality of stabilization agents are uniformly distributed on the substrate or in a capture area on the substrate.

311. The method of claim 306, wherein the capture domain of each of the plurality of capture agents comprises a common nucleic acid sequence.

312. The method of claim 306, wherein the plurality of target nucleic acids comprise mRNA of different genes.

313. The method of claim 306, wherein each of the plurality of stabilization agents comprises the common nucleic acid sequence, and, wherein the common nucleic acid sequence in the stabilization agents is complementary to the plurality of stabilizing sequences in the amplification product.

314. The method claim 306, comprising generating the complement of the captured target nucleic acid at the location of the captured target nucleic acid on the substrate by extending the capture domain or a portion thereof by a polymerase using the captured target nucleic acid as a template.

315. The method of claim 306, comprising removing the captured target nucleic acid or a portion thereof from the substrate.

316. The method of claim 315, comprising generating a complement of the complement of the captured target nucleic acid, and removing the complement of the complement from the substrate for sequence analysis.

317. The method of claim 306, wherein the probe or probe set comprises a 3′ overhang and/or a 5′ overhang upon hybridization to the captured target nucleic acid or complement thereof.

318. The method of claim 306, wherein the probe or probe set is a circular probe or circularizable probe or probe set.

319. The method of claim 318, wherein the circularizable probe or probe set is ligated using the captured target nucleic acid or complement thereof as a template, with or without gap filling.

320. The method of claim 306, wherein the amplification product is a rolling circle amplification product (RCP), wherein the RCP is hybridized to one or more of the plurality of stabilization agents via one or more of the stabilizing sequences in the RCP.

321. The method of claim 306, wherein the target nucleic acid is a first target nucleic acid and the probe or probe set is a first probe or probe set, and the method comprises:
f) contacting the substrate with a second probe or probe set that binds to a second captured target nucleic acid or a complement thereof, wherein the first and second target nucleic acids are different;
g) generating a second amplification product associated with the second probe or probe set, wherein the second amplification product comprises a plurality of stabilizing sequences complementary to a sequence of each of the plurality of stabilization agents; and
h) detecting a signal associated with the second amplification product on the substrate, wherein the location of the signal on the substrate corresponds to the location of the second target nucleic acid in the biological sample.

322. The method of claim 306, wherein the substrate further comprises a plurality of interspersing agents immobilized at interspersed locations on the substrate, and the plurality of interspersing agents do not comprise a capture domain capable of capturing a target nucleic acid of the plurality of target nucleic acids.

323. The method of claim 306, comprising detecting signals associated with multiple amplification products at multiple locations on the substrate.

324. The method of claim 323, wherein the signals detected at the multiple locations on the substrate are optically resolvable from one another.

325. The method of claim 306, wherein the biological sample is a permeabilized tissue sample.
